(19) United States
(12) Patent Application Publication
(10) Pub. No.: US 2019/0321638 A1
(43) Pub. Date: Oct. 24, 2019

(54) METHOD AND APPARATUS FOR PREVENTING OR TERMINATING EPILEPTIC SEIZURES

(71) Applicant: David J. Mogul, Winnetka, IL (US)

(72) Inventor: David J. Mogul, Winnetka, IL (US)

(21) Appl. No.: 16/379,359

(22) Filed: Apr. 9, 2019

Related U.S. Application Data
(63) Continuation-in-part of application No. 15/355,344, filed on Nov. 18, 2016, now Pat. No. 10,252,056.
(60) Provisional application No. 62/257,039, filed on Nov. 18, 2015.

(51) Int. Cl. A61N 1/36; A61B 5/00

(52) U.S. Cl. CPC A61N 1/36064; A61B 5/0478; A61B 5/4094

(57) ABSTRACT

A method and apparatus for preventing or terminating seizures, by stimulating a brain with at least two implanted electrodes, each implanted in a different one of at least two regions of the brain, with a frequency to emulate neuronal synchrony. Upon detecting a potential or actual seizure occurrence, the frequency is electrically applied to the brain upon the detection to preempt or terminate the potential or actual seizure occurrence. Parts in the brain, where brain electrical activity is being measured, that have the highest connectivity are determined by phase-locking coherence. The select subset of areas showing synchrony, i.e., only those with the highest coherence that will best permit inducing a large global synchrony, are stimulated to preempt or terminate the potential or actual seizure occurrence.

### CROSS REFERENCE TO RELATED APPLICATION

This application is a continuation-in-part of U.S. patent application Ser. No. 15/355,344, filed on 18 Nov. 2016, now U.S. Pat. No. 10,252,056, which claims the benefit of U.S. Provisional Patent Application Ser. No. 62/257,039, filed on 18 Nov. 2015. The co-pending parent application is hereby incorporated by reference herein in its entirety and is made a part hereof, including but not limited to those portions which specifically appear hereinafter.

### FIELD OF THE INVENTION

This invention relates generally to a method and apparatus for preventing or terminating seizures and, more particularly, to closed-loop stimulation protocols for preventing or terminating epileptic seizures.

### BACKGROUND OF THE INVENTION

Epilepsy is a brain disorder characterized by recurrent and spontaneous derangements of normal brain activity. More than 50 million people worldwide have epilepsy. Approximately 70% of patients with epilepsy can be successfully treated with anti-epileptic drugs (AEDs). For these cases of intractable epilepsy with seizures either resistant to drug treatment or unsuitable for surgery, alternative therapeutic approaches are needed.

Electrical stimulation has been explored as a potential for benefit in treating epilepsy. With varying degrees of success, several studies have examined the effects of continuous and periodic stimulation for controlling seizures. Results from studies of the NeuroPace™ Responsive Neurostimulator System (RNS) and the stimulation of the anterior nuclei of thalamus for epilepsy (SANTE) demonstrate that deep brain electrical stimulation (DBS) can reduce the occurrence of seizures in select patient populations. In the RNS study, approximately 54% of the patients implanted with the device experience greater than 50% reduction in seizure frequency from pre-implantation period. Most stimulation paradigms in therapeutic devices seek to reduce the frequency of seizure onset but are not specifically tailored to terminate a seizure once ictal activity has initiated simply because past efforts at this goal have not yet shown strong efficacy. Most researchers derive stimulation parameters by trial and error and frequently use as a starting point the experience of DBS for treating movement disorders. There is a continuing need for a stimulation protocol to improve the effectiveness of DBS in stopping epileptic seizures.

### SUMMARY OF THE INVENTION

The invention provides a method and apparatus for preventing or terminating seizures with a stimulation frequency. In embodiments of this invention, the frequency emulates and/or disrupts neuronal synchrony that causes or at least occurs during seizures. The synchrony dynamics of a patient are observed as seizures naturally terminate and are used as an individualized, endogenous mechanism in a method and device for seizure preemption or termination by deep brain stimulation before or during a seizure. In some embodiments, the method includes stimulating a brain with a seizure termination frequency determined from and for a patient at two or more implanted brain electrodes, each implanted in a different one of two or more regions of the brain.

The invention further includes a method and apparatus for preventing or terminating seizures by determining neuronal synchrony between at least two sites of a brain of a patient, and stimulating the brain with at least two implanted electrodes, each implanted in a different one of at least two regions of the brain, with a predetermined seizure termination frequency determined from frequencies measured across a seizure occurrence in the at least two regions of the brain. The neuronal synchrony between the at least two sites of the brain can be determined from brain sites having a highest coherence there between, such as a phase-locking coherence, and the stimulating desirably then occurs at the at least two sites of the brain.

The invention further includes a method of preventing or terminating seizures by monitoring for and/or determining neuronal synchrony between a plurality of sites of a brain of a patient, comparing measurements from and between at least pairs of the plurality of sites to determine at least a pair of sites with a higher electrical coherence than one or more others of the compared sites or pairs, determining a frequency at the higher electrical coherence, and stimulating the brain at the frequency. The higher coherence between the pairs generally means a higher synchronic connectivity between the pairs. The stimulation desirably occurs at the pair of sites with the higher electrical coherence. Also, herein, the use of the term “pairs” includes at least two, and further includes using more than two sites for the determination and/or stimulation.

The invention further includes an apparatus for automatically preventing or terminating seizures. The apparatus includes a neurostimulator with a power supply connected to a stimulation generator that generates electrical stimulation through at least two electrodes for implanting in a patient's brain. The apparatus further includes a control protocol on a non-transitory recordable medium in executable combination with the stimulation generator and adapted to automatically stimulate at least two regions of the brain with a frequency to mimic neuronal synchrony.

The invention further includes a non-transitory computer readable storage medium storing code executable by a processor/controller on an implantable neurostimulator or similar device to perform the method according to this invention. The code can be stored and execute on existing commercial devices or on a new device according to this invention.

In the example embodiments of this invention, multi-site brain dynamics within the circuit of Papez were calculated in a freely-moving chronic rat limbic epilepsy model induced via lithium chloride (LiCl)/pilocarpine i.p injections. Using empirical mode decomposition and coherence analysis, key dynamics were identified as seizures progressed. Synchrony dynamics seen as a seizure naturally terminated were reproduced using exogenous multi-site synchronized stimulation in an effort to stop a progressing seizure. Significantly improved efficacy of the stimulation at terminating seizures was found when the stimulation frequency and location of multi-site synchronized stimulation matched the endogenous synchrony dynamics observed during natural termination in the animal.

### BRIEF DESCRIPTION OF THE FIGURES

FIG. 1 generally illustrates an implanted apparatus according to one embodiment of this invention.

FIG. 2 shows that synchrony dynamics of oscillators in different structures in a rat brain during natural termination are similar between spontaneous and evoked seizures.

FIG. 3 shows that synchrony dynamics of oscillators during natural seizure termination in the rat brain varies from subject to subject.

FIG. 4 shows therapeutic stimulation at the target frequency observed by synchrony analysis terminates evoked seizures faster than stimulation at non-target frequencies.

FIG. 5 shows endogenous synchrony dynamics within circuit of Papez during temporal lobe seizures.

FIG. 6 displays electrographic activity containing evoked seizures recorded from the intracranial electrode located in the CA3 region of the right hippocampus from four rats.

FIG. 7 summarizes responses to therapeutic DBS as a function of stimulation frequency during temporal lobe seizures.

FIG. 8 shows that endogenous synchrony dynamics and frequency-sensitivity of therapeutic stimulation are similar in spontaneous vs. evoked seizures.

FIG. 9 is Fourier-derived spectrograms in rats with different endogenous termination dynamics.

FIG. 10 shows that sensitivity of efficacy to DBS stimulation locations depends on where coherence at natural termination occurs.

FIG. 11 is a table showing the number of seizures that have undergone experimental protocols for each of nine animal subjects.

FIG. 12 is a conceptual front-end of the proposed phase-synchrony analysis and phase connectivity analysis for Example III.

FIGS. 13A and 13B show selections of IMF (bandwidth of interest) for DBS.

FIGS. 14A-D shows phase-synchrony and phase-connectivity analysis for selecting DBS locations and frequency in example Patient-1 with TLE. FIG. 14A shows an anatomical position of depth electrodes. FIG. 14B shows a 3-min, iEEG recording containing a seizure. FIG. 14C shows temporal changes of the normalized meanλ_{1:60%} value, after smoothing with a 5-s moving average filter, for IMFs extracted from all channels. FIG. 14D shows phase-connectivity analysis for the selected IMF, (IMF5).

FIGS. 15A-D show phase-synchrony and phase-connectivity analysis for selecting DBS locations and frequency in example Patient-2 with ETE. FIG. 15A shows an anatomical position of depth electrodes. FIG. 15B shows a 4-min iEEG recording containing a seizure. FIG. 15C shows temporal changes of the normalized meanλ_{1:60%} value, after smoothing with a 5-s moving average filter, for IMFs extracted from all channels. FIG. 15D shows phase-connectivity analysis for the selected IMF, (IMF2).

FIGS. 16A-D show phase-synchrony and phase-connectivity analysis for selecting DBS locations and frequency in example Patient-3 with ETE. FIG. 16A shows anatomical position of depth electrodes. FIG. 16B shows a 4-min iEEG recording containing a seizure. FIG. 16C shows temporal changes of the normalized meanλ_{1:60%} value, after smoothing with a 5-s moving average filter, for IMFs extracted from all channels. FIG. 16D shows a phase-connectivity analysis for the selected IMF, (IMF2).

### DESCRIPTION OF THE INVENTION

A general object of the invention is to use endogenous multisite brain dynamics to engineer effective closed-loop stimulation protocols for automatically preventing or terminating epileptic seizures. The invention involves stimulating a brain with at least two implanted electrodes, each implanted in a different one of at least two regions of the brain, with a frequency to emulate or otherwise promote neuronal synchrony. This invention includes a method and algorithm for providing deep brain electrical stimulation that are based on real-time brain activity of individuals with chronic recurrent seizures that are emblematic of epilepsy. The method of this invention is desirably implemented, without limitation, in a medical device that would be implanted in a human with chronic epilepsy to automatically carry out the detecting and preventing/terminating of a seizure event.

The invention includes a method that prevents and/or terminates seizures by determining and continually, automatically monitoring for occurrences of neuronal synchrony or other seizure indicator in regions of the patient's brain. Such synchrony has been found to precede and/or otherwise occur during a seizure event. The implanted electrodes can be used to identify synchronization and seizure between two or more regions of the brain during a seizure. In embodiments of this invention, frequencies of the seizure for the patient are measured and analyzed to determine a seizure termination frequency. The seizure termination frequency is determined from seizure frequencies measured across a seizure occurrence in the at least two regions of the brain. In preferred embodiments of this invention, the seizure termination frequency is obtained from electrophysiological dynamics of the brain measured at or near an end of a seizure within the brain. In this way, seizure dynamics (e.g., neuron frequencies and/or synchrony locations) can be determined for each individual patient. Once an appropriate frequency is determined for a patient, the frequency is electrically applied to the appropriate regions of the brain upon the detection of a potential or actual further seizure event to prevent or terminate the seizure.

In embodiments of this invention, the frequency promotes an evolving synchronization between structures in the at least two regions of the brain to preempt or otherwise terminate a seizure. The electrical behavior of parts of an epileptic human brain show increased overall synchronization (hypersynchrony) as seizures terminate. In embodiments of this invention, the parts in the brain (where brain electrical activity is being measured) that have the highest connectivity are found, such as measured by phase-locking coherence. Only a select subset of areas showing synchrony, i.e., only those with the highest coherence that will best permit inducing a large global synchrony, are stimulated. Another way of thinking about this is that sites in the brain that are well-connected to other key sites will act as connection nodes to help drive this increased synchrony. By doing this, the number of sites that need to be stimulated in the brain are reduced, using the frequency that shows this high coherence/connectivity in order to create the therapeutic synchrony seen at termination.

There are two disease-related pathological events that could be targeted with this new stimulation process. One would be stimulation to terminate a seizure. That is to say, the stimulation strategy of embodiments this invention would be activated once a seizure has been detected and then stimulation would be applied using the specific stimulation protocol tailored to an individual epilepsy patient that would apply multi-site stimulation to stop the seizure from continuing. The second event would be to prevent brain electrophysiological behavior from progressing to a full-blown seizure. Embodiments of the invention utilize key brain electrophysiological dynamics that arise just as full-blown seizures occur. In a similar strategy to termination, stimulation is applied to stop those dynamics from progressing by disrupting an evolving synchronization between structures in the brain tailored to a patient's individual brain electrical dynamics as seizures evolve.

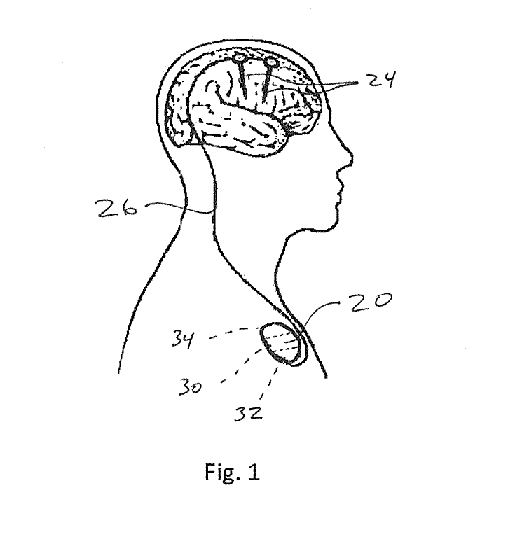

FIG. 1 shows an implanted apparatus 20 according to one embodiment of this invention. The apparatus includes a neurostimulator 22 that is implanted in the patient's chest cavity. The neurostimulator 22 includes an internal power supply 30 to power a hardware/software based stimulation generator 32 to automatically generate electrical stimulation through two electrodes 24 connects by wires 26 disposed under the skin. The two electrodes are implanted within the brain; in two separate regions of the brain, such as, without limitation, each disposed in a different hemisphere of the brain.

The invention desirable has at least two electrodes, but preferably can include more than two electrodes, each in a different region of the brain. The use of three, four, six, eight, etc. electrodes can increase the ability to automatically determine synchronization events between two or more regions of the brain, thereby increasing the efficiency and/or effectiveness of the invention. Suitable electrodes, such as with sensor features for measuring and monitoring brain frequencies, are commercially available.

The apparatus 20 further includes a control protocol on a non-transitory recordable medium, such as within the neurostimulator 22, and in executable combination with the stimulation generator. The control protocol includes coded instructions to operate the electrodes and stimulate the two (or more) regions of the brain with a frequency to disrupt neuronal synchrony. The frequency supports and/or promotes an evolving synchronization between structures in the two regions of the brain to preempt or terminate a seizure. The frequency is synchronized to a predetermined seizure termination frequency of the at least two regions of the brain.

The apparatus 20 of embodiments of this invention further includes a detector 34 adapted to determine any occurring neuronal synchrony, and in doing so determines a potential or actual seizure occurrence. The detector can be hardware and/or coded instructions within the neurotransmitter 20 in combination with a sensor functionality of the electrodes 24. In embodiments of this invention, the detector 34 and electrodes 24 are used to monitor patient seizures and determine a predetermined seizure termination frequency for use in preempting or terminating future seizures according to this invention. In embodiments of this invention, the predetermined seizure termination frequency is obtained from electrophysiological dynamics of the brain measured at or near an end of a seizure within the brain.

There are currently two medical devices—one on the market and the other being clinically tested—that are implanted in humans with epilepsy that apply electrical stimulation to the brain in order to try to reduce seizure frequency. These devices (i.e., The Neuropace RNS™ system and the Medtronic SANTE™ device, respectively) do not use brain electrical dynamics to formulate stimulation protocols. In contrast, the invention described herein uses brain electrical dynamics tailored to individual patient brain dynamics as seizures evolve to fashion much more targeted stimulation protocols directed at pathological endogenous electrical activity in the brain. The method of this invention can be implemented on these known devices by loading the devices with the suitable software instructions.

Embodiments of this invention for treating epilepsy with deep brain stimulation (DBS) apply electrical stimulation in predetermined protocols (i.e., the timing of stimulation, the frequency, duration and location of stimulation) that are largely independent of the electrophysiological behavior occurring within the brain. The focus of the stimulation paradigm incorporated in this invention has been to apply excitatory stimulation with the goal of disrupting the evolution of onset of a seizure in a largely unspecified way. The invention uses analytical techniques to find endogenous synchronization dynamics related to seizures to formulate new deep-brain stimulation protocols to treat seizures. This new process has identified critical synchronization events in the brain that inform how to tailor stimulation to interfere with seizure evolution.

DBS is a potentially potent means for disrupting the aberrant rhythms that arise during a seizure. However, current DBS strategies typically employed are formulated a priori and do not reflect dynamics within the brain during ictogenesis which may severely limit stimulation efficacy. According to embodiments of this invention, DBS can be improved using endogenous dynamics to inform stimulation protocols. As discussed below, multi-site brain dynamics within the circuit of Papez was calculated in a chronic rat limbic epilepsy model. Stimulation/recording electrodes were placed in the CA3 region of both hippocampi and in the anteromedial nucleus of the thalamus. Deconvolution of signals using empirical mode decomposition and coherence analysis was used to identify key dynamics as seizures progressed. Synchronization of field potentials across sites occurred as both spontaneous and evoked seizures naturally terminated. The location and frequency of synchrony varied between subjects suggesting that endogenous rhythms during natural seizure termination may vary in humans as well. DBS efficacy was significantly more effective at stopping seizures when the frequency of multisite synchronized stimulation reflected endogenous synchrony dynamics observed in each subject. Thus, tailoring DBS protocols to individual endogenous rhythms that may represent how brains naturally resolve epileptic seizures can play a critical role in improving overall efficacy of this potentially important therapy.

As stated above, an object of this invention is to determine the most effective sites and frequency for stimulation to effect the critical synchrony to terminate a seizure personalized DBS to each patient. Embodiments of this invention use information from analyzing brain electrical data recorded from multiple locations within the brain, such as is generally performed as an evaluation for surgery in patients with drug-resistant epilepsy. This electrical data, recorded from multiple locations, can be analyzed for global synchrony, to see what happens to the relative behavior between brain sites as seizures evolve. As seizures initially start, global desynchronization (i.e., a decrease in synchronization among all of the sites) is generally seen. As seizures naturally terminate, big increases in global synchrony are seen. The invention promotes the increase in synchrony across a broad section of the brain in a personalized (i.e., which may have characteristics that are different for different patients with epilepsy) and also efficient fashion. To address this goal, embodiments of this invention calculate comparisons of the connectivity between all the different pairs of sites where electrical behavior is being recorded (also called “bivariate coherence”). The higher the coherence, the stronger the connectivity. The brain sites (also called nodes) that are most strongly connected within the entire recording regions within the brain are determined, and the frequency at which this strong coherence occurs is also determined or calculated. From this analysis, the sites and the frequency for each patient at which DBS should be applied is provided. In other words, this represents a methodology by which one can derive what is believed to be the best stimulation protocol to induce global synchrony in the brain and hence, the strongest efficacy for terminating or preventing seizures.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### EXAMPLES

### Example I

Experiments were performed on male Sprague-Dawley rats. Experimental protocols were conducted in accordance with National Institute of Health instructions for the care and use of laboratory animals. The rats had unlimited access to food and water. They were maintained in individual cages with 12-h light/dark cycles, with the light on from 6 AM-6 PM.

### Surgery Procedures and Seizure Induction

Rats were anesthetized with a mixture of Ketamine (80 mg/kg) and Xylazine (10 mg/kg) delivered intra-peritoneally and then fixed within a stereotaxic apparatus (KOPF Model 900, CA, USA). The plane of anesthesia was continually assessed by reaction to a toe-pinch stimulus and corneal eye-blink reflex. Anesthesia was maintained with boosters containing Ketamine (20 mg/kg) delivered intramuscularly. A midline incision was made from the bridge of the nose to the posterior end of the cranium. Stereotaxic targets were calculated using a stereotaxic rat brain atlas. Lambda, Bregma and Sagittal sutures were used as landmarks to navigate to the desired stereotaxic points. The skull was perforated using a high speed stereotaxic drill (Micromotor Drill, Stoelting Co, IL USA) with 1.2-2 mm diameter drill tips. Six small burr holes were drilled: three were for the positioning of anchor screws and three for the placement of electrodes. Bipolar stainless steel electrodes (E363-1-2TW-SPC; Plastics One, Roanoke, Va., USA) were implanted into the CA3 regions of the bilateral hippocampi (−3.5 mm bregma, +2.8 mm lateral, 3.7 mm deep) and the left anteromedial thalamus (−1.56 mm bregma, 1.0 mm lateral, 6.2 mm deep). The electrode sockets were inserted into a six-channel electrode pedestal (MS363; Plastics One) and the whole assembly was fixed to the skull using acrylic dental cement. After the cement dried (several minutes), the scalp was sutured closed and an electrode dust cap was screwed onto the pedestal. A week after surgery, the rats underwent seizure induction. Lithium chloride (127 mg/kg, i.p.) was injected 19-24 hours prior to pilocarpine administration. Scopolamine (1 mg/kg, i.p.) was administered 30 minutes before pilocarpine administration. Repeated doses (≤3) of pilocarpine (10 mg/kg, i.p.) were given to the rat every 30 minutes until the emergence of the first stage 4/5 seizure. Diazepam (10 mg/kg, i.p.) was injected 90 minutes after the onset of status epilepticus to quench ictal activity.

### Stimulation and EEG Acquisition

A few weeks after seizure induction, local field potentials (LFPs) were recorded with an amplification per channel of 5000 and bandpass filtered (1-1000 Hz) using a Grass amplifier (QP511; Grass Technologies, West Warwick, R.I.). The signals were digitized at 2000 samples/second with a 32-bit A/D converter using an ADwin-light-16 unit (Jager GmbH, Lorsch, Germany). Spontaneous and evoked seizures were recorded from the animals. Seizures were evoked using 200 Hz square pulses (biphasic, 1 ms pulse width, and 80 μA) that lasted for 10 seconds. Software developed in-laboratory that employed Visual C# (Microsoft Corp., Seattle, Wash.) and MATLAB (The Mathworks, Inc, Natick, Mass.) compilers was used to acquire and analyze electrophysiological data. Therapeutic DBS was delivered by an electrical stimulator (WPI A359, Sarasota, Fla., USA). Square pulses of varying frequencies (biphasic, 1 ms pulse width, and 80 μA) were tested for their effectiveness in terminating the evoked seizures. For all subjects, video recordings were continuously maintained during all experimental protocols to provide behavioral correlates to electrographic activity.

### Electrographic Analysis

LFPs from the bilateral hippocampi and the anteromedial nucleus of thalamus were decomposed into a series of intrinsic mode functions (IMFs) using the method of empirical mode decomposition (EMD). The instantaneous phase of each IMF was calculated using the Hilbert analytic signal method. The IMFs were clustered using eigenvalue-eigenvector clustering. Phase synchronization of the topmost cluster for each analysis window was assessed using the phase locking value. The significance of the observed phase synchrony was evaluated using surrogates and the frequencies of the IMFs that showed significant phase locking were estimated from their phase information. A more detailed explanation of this process can be found in T. Sobayo, et al., “Synchrony Dynamics Across Brain Structures In Limbic Epilepsy Vary Between Initiation And Termination Phases Of Seizures,” IEEE Trans Biomed Eng, 2013; 60:821-829, herein incorporated by reference.

### Synchrony Dynamics Observed in Spontaneous and Evoked Seizures

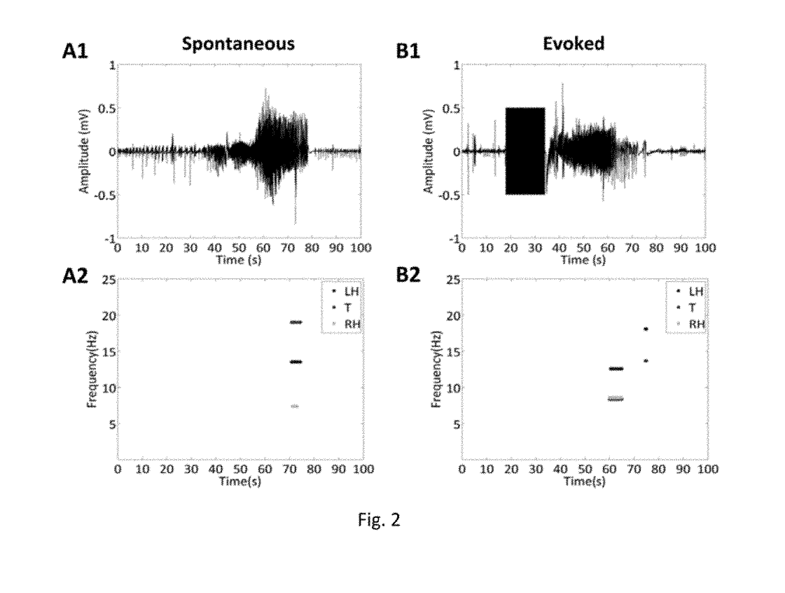

LFPs were recorded in awake, free-moving rats from three locations implicated in limbic seizures as described above. Deconvolutions of the signals using empirical mode decomposition and coherence analysis were used to identify endogenous synchrony dynamics. FIG. 2 displays the LFP during either a spontaneous (panel A; left) or an evoked (panel B; right) seizure recorded from the same experimental animal. Panels A1 and B1 show 100 seconds of electrographic activity recorded from the intracranial electrode located in the CA3 region of the right hippocampus. Panels A2 and B2 show the corresponding synchrony analysis depicting the frequencies of the phase locked oscillators from the three recording sites. Note that the frequency of synchronization during natural seizure termination was similar for both spontaneous and evoked seizures. This result was common indicating the mode of seizure induction did not alter the observed synchrony at seizure termination. Evoked seizures and synchrony analysis for two other experimental animals is shown in FIG. 3. In the three animals shown, a period of synchronous locking of oscillators was seen at some or all of the three recording locations as the seizures terminated. However, the location and frequency of the synchrony varied between the animals. Since the epileptogenic induction protocols for these chronic animals were always the same, it indicates some heterogeneity in the seizure induction network. This difference may also reflect potential differences in seizure dynamics in human patients that may at least partly underlie differential efficacy to simple DBS protocols between patients. Hence, DBS protocols were tailored to individual subject synchrony dynamics and then tested to assess their efficacy in stopping evoked seizures in each subject.

### Frequency of Multisite Synchronized Stimulation Affects the Efficacy of DBS

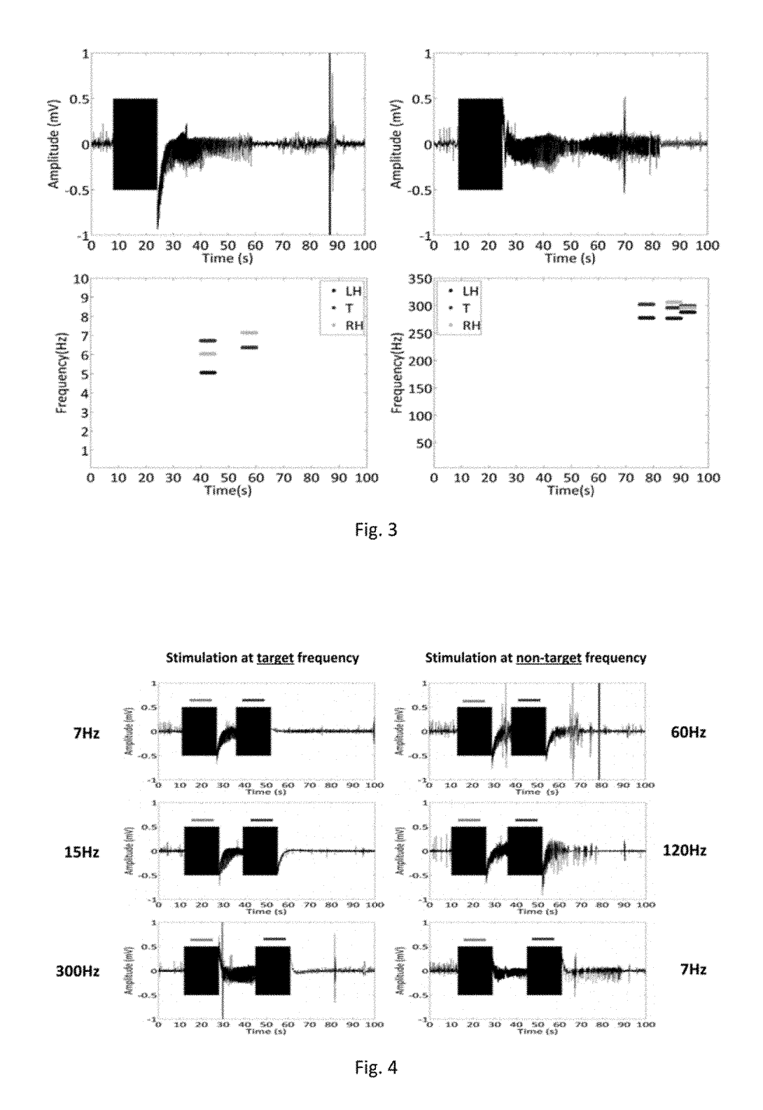

Seizures were evoked in each experimental subject as described above. The parameters for the DBS protocols were the same (i.e., pulse width, amplitude, duty cycle) except for the location and frequency of stimulation. For each animal, the frequency of the stimulation was chosen to be either the target frequency observed by the synchrony analysis to occur at natural termination or a frequency different from the target frequency. FIG. 4 displays 100 seconds of electrographic activity containing evoked seizures recorded from the intracranial electrode located in the CA3 region of the right hippocampus from three different rats. In each case, the left panels show therapeutic stimulation at the target frequency that matched the synchrony at natural termination for that animal. The right panels show therapeutic stimulation at frequencies other than the target frequencies. Stimulation at the frequencies reflecting the endogenous synchrony dynamics observed in each subject rapidly reset the LFP waveform back to its preictal state while stimulation at other frequencies did not appear to terminate the seizures since ictal activity continued for up to a minute after the therapeutic stimulation ended.

The effectiveness of terminating evoked seizures in the animals was measured using two criteria: (a) the time it took for the seizure to end after therapeutic stimulation was halted, and (b) an efficacy measure which took into account both the time it took for the seizure to stop after stimulation and the delay between the induction and therapeutic stimulation protocols. The target frequencies stop the seizures significantly faster than the control frequencies in most cases. It is interesting to note that in all three animals, the target frequencies stop the seizures significantly faster than the stimulation at 120 Hz (which is in a frequency range commonly used in stimulation experiments in rats). For the target frequencies, the time it took for seizures to stop were 14.70±3.41 s, 12.71±3.25 s, and 6.36±3.04 s for rat 1, 2, and 3 respectively; while the times for stimulation at 120 Hz were 43.83±7.35 s, 22.17±3.11 s, and 20.29±4.09 s for rat 1, 2, and 3 respectively (p=0.002, p=0.04, p=0.007). Stimulation efficacy measure also showed a similar trend where the target frequencies were more effective than the non-target frequencies.

### Conclusion

The goal of this research was to investigate whether reproducing endogenous synchrony dynamics observed during natural seizure termination through exogenous electrical stimulation improved the efficacy of reverting epileptic seizures with DBS. The key findings of this study were: (1) the efficacy in reverting seizures via DBS was significantly improved when the stimulation frequency matched the target frequency obtained from synchrony analysis, and (2) the efficacy in reverting seizures was significantly improved when the multisite stimulation locations match the ones where endogenous synchrony emerged as seizures terminated.

Most studies in the use of DBS for epilepsy treatment derived stimulation parameters by trial and error. When it comes to the stimulation frequency, there exists competing rationale for selecting low, medium, or high. This might explain the varying degrees of success shown in several studies of using DBS to treat recurrent seizures. Since the endogenous dynamics of the seizures were not typically considered in the selection of the DBS frequency, differences in endogenous dynamics between subjects may lead to significantly different efficacy assessments of a single stimulation frequency. The data indicate that the time taken for a seizure to stop was significantly lower when the stimulation frequency matched that from the endogenous synchrony analysis of each subject. The target frequency for each subject varied significantly between subjects. For example, the 7 Hz frequency which was most effective in subject 1 was ineffective for subject 3. The location of the stimulation also appeared to be an important factor when it came to stimulation efficacy. For all three subjects, the hippocampus appears to be an important location for stimulation. Thalamic stimulation alone was less effective than bilateral hippocampal stimulation or a combination of both. The exact nature of the anti-convulsant action of electrical stimulation is not fully understood. Generally, accepted theories center around the following hypotheses: (a) preferential release of inhibitory neurotransmitters due to stimulation and/or (b) depolarization block, in which the stimulated neurons are inactivated because of stimulus-induced membrane hyperpolarization. It has also been suggested that stimulation with a high frequency has a lesion-like effect (depolarization block), while low frequency stimulation induces long-term depression (LTD) of stimulated neurons in the peri-electrode space. However, since the target frequencies in some cases were quite low (<20 Hz), it's unlikely that this depolarization block after 10 sec of therapeutic stimulation would have been significant.

### Example II

Patterns of phase synchrony have been observed between three subcortical nuclei: bilateral hippocampi and the left anteromedial thalamus in the circuit of Papez only during the initiation and termination phases of seizures. This is consistent with other studies that looked at synchronization during experimental animal seizures as well as during termination in human seizures. In this study, multi-site brain dynamics within the circuit of Papez were calculated in a freely-moving chronic rat limbic epilepsy model. Using empirical mode decomposition and coherence analysis, key dynamics were identified as seizures progressed. Synchrony dynamics seen as a seizure naturally terminated were reproduced using exogenous multi-site synchronized stimulation in an effort to stop a progressing seizure. Significantly improved efficacy of the stimulation at terminating seizures was found when the stimulation frequency and location of multi-site synchronized stimulation matched the endogenous synchrony dynamics observed during natural termination in the animal.

This study investigated how the efficacy of DBS could be improved using endogenous dynamics to inform stimulation protocols. Multi-site brain dynamics within the circuit of Papez were calculated in a chronic rat limbic epilepsy model induced via LiCl/pilocarpine i.p. injections. Stimulation/recording electrodes were placed in the CA3 region of left and right hippocampi and the anteromedial nucleus of left thalamus. Deconvolution of local field potentials using empirical mode decomposition (EMD) and phase synchrony analysis revealed multisite coherence as seizures approached natural termination that could not be detected with Fourier analysis. Multisite stimulation used charge-neutral biphasic square waves at frequencies observed during naturally termination.

Synchronization of electrical activity across sites occurred as both spontaneous and evoked seizures naturally terminated. Further, the location and frequency of the synchrony varied between subjects but was stable in time within each animal. DBS efficacy was significantly more effective at rapidly stopping seizures when the frequency and location of multi-site stimulation reflected the endogenous synchrony dynamics observed in each subject as seizures naturally terminated.

These results strongly support the approach of tailoring DBS protocols to individual endogenous rhythms that may represent how brains naturally resolve epileptic seizures can significantly improve the overall efficacy of this potentially important therapy.

### Material and Methods

Experiments were performed on male Sprague-Dawley rats. The experimental protocols were conducted in accordance with the National Institute of Health instructions for the care and use of laboratory animals. The rats had unlimited access to food and water. They were maintained in individual cages with 12-h light/dark cycles, with the light on from 6 AM to 6 PM. In nine rats, evoked and spontaneous seizures were collected and used in testing the efficacy of stimulation. Three out of the nine rats were removed from the study because the head caps became partially detached before completion of all experimental protocols.

### Surgery Procedures and Seizure Induction

Rats (290-350 gm) were anesthetized with a mixture of Ketamine (80 mg/kg) and Xylazine (10 mg/kg) delivered intra-peritoneally and then fixed within a stereotaxic apparatus (KOPF Model 900, CA, USA). The plane of anesthesia was continually assessed by reaction to a toe-pinch stimulus and corneal eye-blink reflex. Anesthesia was maintained with boosters containing Ketamine (20 mg/kg) delivered intramuscularly. A midline incision was made from the bridge of the nose to the posterior end of the cranium. Stereotaxic targets were calculated using a stereotaxic rat brain atlas. Lambda, Bregma and Sagittal sutures were used as landmarks to navigate to the desired stereotaxic points. The skull was perforated using a high speed stereotaxic drill (Micromotor Drill, Stoelting Co, IL USA) with 1.2-2 mm diameter drill tips. Six small burr holes were drilled: three were for the positioning of anchor screws and three for the placement of electrodes. Bipolar stainless steel electrodes (E363-1-2TW-SPC; Plastics One, Roanoke, Va., USA) were implanted into the CA3 regions of the bilateral hippocampi (−3.5 mm bregma, ±2.8 mm lateral, 3.7 mm deep) and the left anteromedial thalamus (−1.56 mm bregma, 1.0 mm lateral, 6.2 mm deep). The electrode sockets were inserted into a 6 channel electrode pedestal (MS363; Plastics One) and the whole assembly was fixed to the skull using acrylic dental cement. After the cement dried (several minutes), the scalp was sutured closed and an electrode dust cap was screwed onto the pedestal.

One week after surgery, the rats underwent seizure induction. Lithium chloride (127 mg/kg, i.p.) was injected 19-24 hours prior to pilocarpine administration. Scopolamine (1 mg/kg, i.p.) was administered 30 minutes before pilocarpine administration. Repeated doses (≤3) of pilocarpine (10 mg/kg, i.p.) were given to the rat every 30 minutes until the emergence of the first stage 4/5 seizure. Diazepam (10 mg/kg, i.p.) was injected 90 minutes after the onset of status epilepticus to quench ictal activity.

### Stimulation and EEG Acquisition

Spontaneous seizures typically arose 4-8 weeks after induction. Local field potentials (LFPs) were recorded with an amplification per channel of 5000 and bandpass filtered (1-1000 Hz) using a Grass amplifier (QP511; Grass Technologies, West Warwick, R.I.). The signals were digitized at 2000 samples/second with a 32-bit A/D converter using an ADwin-light-16 unit (Jager GmbH, Lorsch, Germany). Spontaneous and evoked seizures were recorded from the animals. Seizures were evoked using 200 Hz square pulses (biphasic, 1 ms pulse width, and 80 μA) applied to the bilateral hippocampi that lasted for 10 seconds. Software developed in-laboratory which employed Visual C# (Microsoft Corp., Seattle, Wash.) and MATLAB (The Mathworks, Inc, Natick, Mass.) compilers was used to acquire and analyze electrophysiological data. Therapeutic DBS was delivered by an electrical stimulator (WPI A359, Sarasota, Fla., USA). Square pulses of varying frequencies (biphasic, 1 ms pulse width, and 80 μA) were tested for their effectiveness in terminating the evoked seizures. For all subjects, video recordings were continuously maintained during all experimental protocols to provide behavioral correlates to electrographic activity.

### Electrographic Analysis

Local field potentials (LFPs) represent a sum of dendritic activity that may be inhibitory or excitatory; hence, these waveforms are necessarily multi-component. Since phase synchrony measures rely on prior extraction of the phase information from the time series, one critical step in synchrony analysis is phase determination. For a non-stationary signal, one method of extracting phase values instantaneously is the Hilbert analytic signal method for mono-component signals. Thus, a prior step to phase calculation will involve some form of filtering of the data. Almost all of the commonly employed decomposition algorithms assume something about the waveform shape or frequency bandwidth of interest a priori.

Empirical mode decomposition (EMD) allows one to filter a multi-component signal into a series of oscillators representing the (adaptively determined) characteristic time-scales of the individual components without a priori assumptions of linearity or stationarity. LFPs from the bilateral hippocampi and the anteromedial nucleus of thalamus were decomposed into a series of intrinsic mode functions (IMFs) using EMD. Instantaneous phase of each IMF was calculated using the Hilbert analytic signal method and the IMFs were clustered using eigenvalue-eigenvector clustering which provided a way of ranking different sets of synchronous oscillators. Phase synchronization of the topmost cluster for each analysis window was quantitatively assessed. The significance of the observed phase synchrony was evaluated using Fourier shuffled surrogates and the frequencies of the IMFs that showed significant phase locking were estimated from their phase information. A more detailed explanation of this process can be found in Blenkinsop et al., “The dynamic evolution of focal-onset epilepsies—combining theoretical and clinical observations,” Eur. J. Neurosci. 2012; 36:2188-2200, and Fine et al., “Assessing instantaneous synchrony of nonlinear nonstationary oscillators in the brain,” J. Neurosci. Methods 2010; 186:42-51.

The average frequency between oscillators from different locations that showed significant phase locking was used as the target frequency for therapeutic stimulation aimed at stopping ongoing pathological electrographic activity. Other frequencies (derived from the literature) and stimulation locations were used as controls to assess the comparative efficacies of the stimulation protocols at reverting epileptic seizures.

Efficacy of therapeutic stimulation (TS) was assessed with respect to the duration of a seizure relative to those that naturally resolved with no therapeutic stimulation (NS). This relationship is provided by:

Efficacy=1−A(1−e^{-t}^{_{TS}}^{/t}^{_{NS}}),

where A=1/1−e⁻¹ such that when seizure duration following therapeutic stimulation is the same as with no such stimulation, Efficacy=0. Stimulation that immediately disrupted seizures have an Efficacy=1 and therapeutic stimulation that produced seizure durations longer than that of control (i.e., NS) produced negative efficacies.

### Statistical Analysis

All data were presented as mean±standard error of the mean. SigmaStat (Systat Software Inc., USA) was employed for statistical analysis. One way ANOVA or Kruskal-Wallis one way ANOVA on ranks was used to determine the effectiveness of DBS protocols depending on conditions of normality. A value of p<0.05 was considered statistically significant.

### Frequency of Multi-Site Synchronized Stimulation Affects the Efficacy of DBS

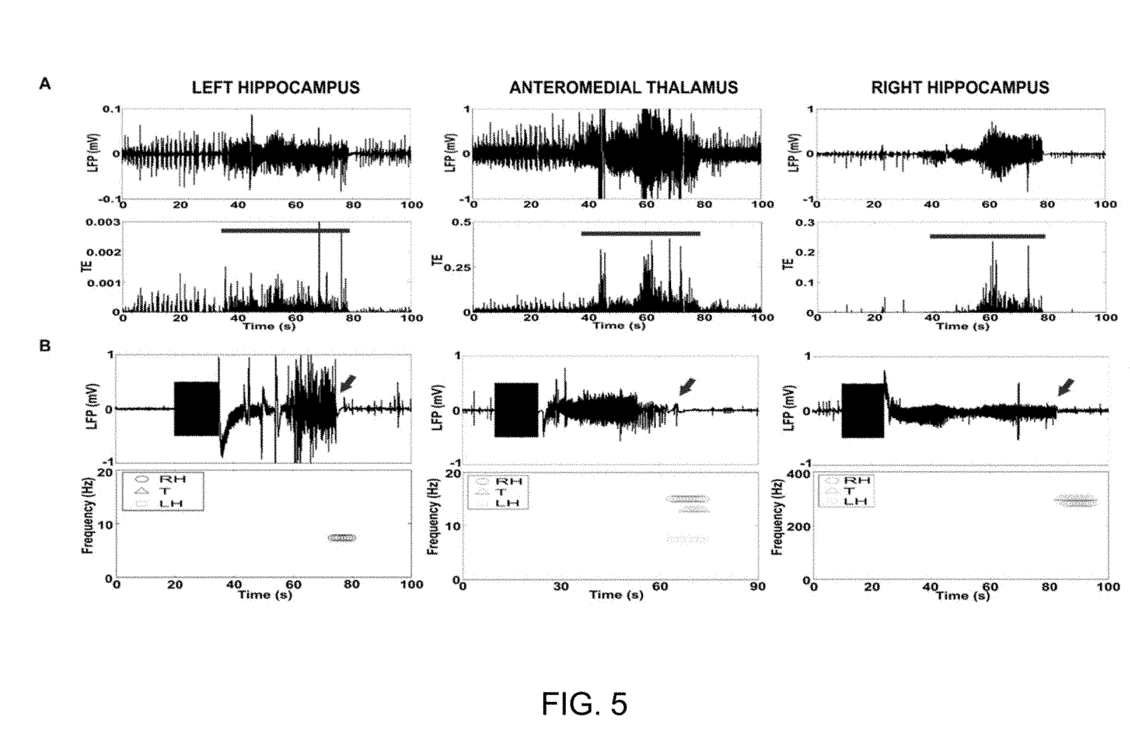

Brain dynamics were calculated at three sites within the circuit of Papez in a chronic rat limbic epilepsy model. FIG. 5 displays local field potentials (LFPs) recorded in awake, freely-moving rats during a spontaneous seizure (panel A). Teager Energy (TE), which depends on both amplitude and frequency of a time series signal, was an excellent indicator of seizure onset and offset that correlated well with behavioral indicators of seizure-induced motor activity (Racine scale ≥3). A threshold of the mean of the TE plus five times the standard deviation was used as criteria for indicating valid seizure activity. Panel B shows electrographic activity during evoked seizures recorded from the intracranial electrode location in the CA3 region of the right hippocampus in three different rats. In all three animals, periods of synchronous locking of oscillators at some or all of the three recording locations as seizures naturally terminated were found. This synchrony was only observed at or near the end of a seizure. The location and frequency of the synchrony varied between animals but was stable over time for each animal (measured up to 5 months post-induction). In nine animals, five different frequencies were observed at natural termination. These termination coherence frequencies were (numbers of animals in parentheses): 7 Hz (3), 15 Hz (2), 120 Hz (2), 250 Hz (1), and 300 Hz (1). Natural termination coherence frequencies were measured a minimum of 2 times for each animal.

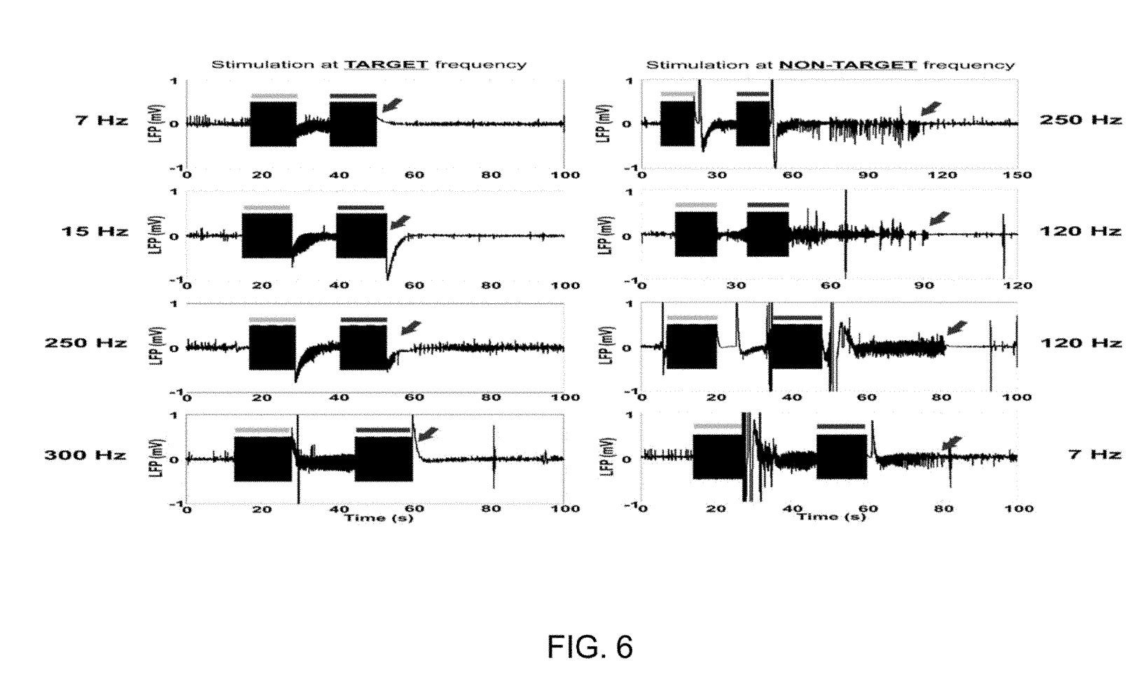

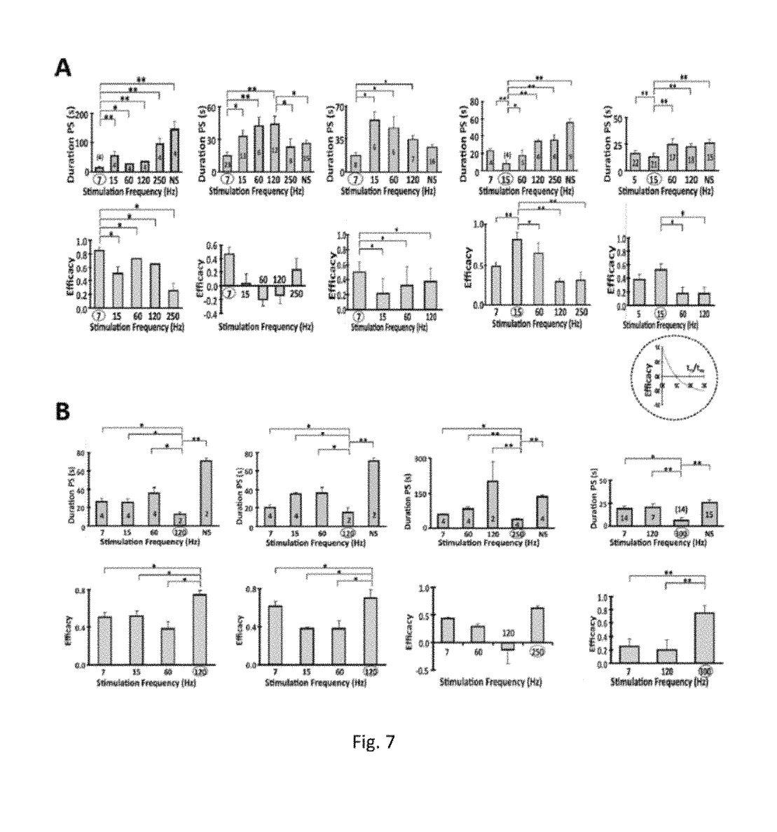

FIG. 6 displays electrographic activity containing evoked seizures recorded from the intracranial electrode located in the CA3 region of the right hippocampus from four rats. In each case, the left panels show therapeutic stimulation at the target stimulation frequencies defined as the frequency of endogenous synchrony at natural seizure termination for that subject. The right panels show therapeutic stimulation at frequencies other than the target frequencies (non-target). Stimulation at the target frequency was significantly faster than non-target frequencies at terminating ongoing seizure activity. FIG. 7 shows the time it took seizures to stop at different stimulation frequencies (upper) and the relative efficacy of stimulation frequencies (lower) for nine different animals with either low target frequencies (panel A) or high target frequencies (panel B).

The effectiveness of terminating evoked seizures in the animals was measured using two criteria: (a) the time it took for the seizure to end after therapeutic stimulation was halted, and (b) an efficacy measure which assessed the duration of a seizure relative to those that naturally resolved with no therapeutic stimulation. The details of the latter measure can be found in the Methods section. Stimulating at the endogenous synchrony frequency observed at natural termination for that animal was significantly better at stopping an ongoing seizure than at other frequencies (p-values as shown in FIG. 7 for each subject). Note that for one of the animals with target frequencies 7 and 250 Hz each, stimulation at non-target frequencies actually produced negative efficacies indicative of stimulation that actually lengthened seizure duration beyond that observed without any therapeutic stimulation (NS).

### Synchrony Dynamics Observed in Spontaneous and Evoked Seizures

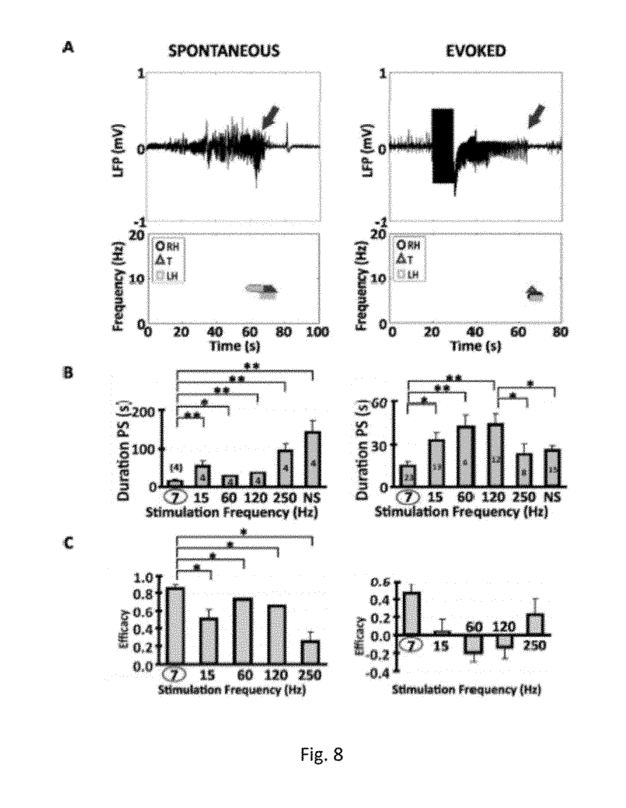

Because the rate of occurrence of spontaneous seizures could vary greatly between animals, seizures were often evoked by a 10 second induction stimulation in these experiments, as described above. The question arises as to whether synchrony dynamics at termination and/or the efficacy of the DBS protocols differed between spontaneous vs. evoked seizures. In fact, analysis comparing both the dynamic of synchrony at natural termination and the dependence of the efficacy of therapeutic DBS (FIG. 8) within subjects indicated that the termination dynamics were largely independent of the mechanism of seizure induction suggesting that the synchrony at natural termination was a function of the underlying network within the brain rather than the mechanism of seizure onset (p=0.004 for spontaneous seizures and p<0.001 for evoked seizures). The significantly better efficacy of stimulation at the natural termination frequency was observed in both induction models. Note that nontarget stimulation protocols in the evoked seizures produced negative efficacies indicating that nontarget stimulation frequencies actually lengthened seizure durations compared to controls.

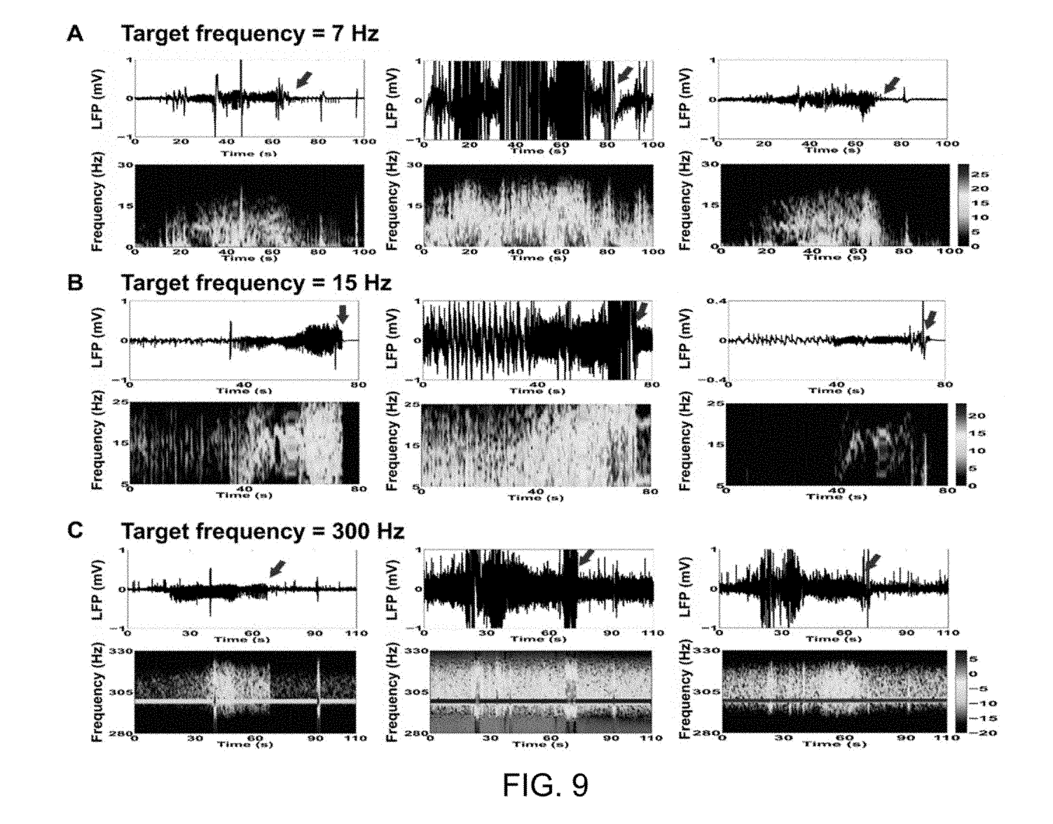

Coherence analyses of the LFP signals at each of the three brain locations were conducted after signals were first decomposed using EMD. Because EMD neither assumes linearity nor stationarity of the underlying signals, the question arises as to whether the simpler and more common decomposition method available via Fourier analysis would have resulted in a similar finding. The size of the temporal window used in Fourier analysis depends, in part, on the critical frequency characteristics of the signal being analyzed—the lower the frequency components being analyzed, the longer the window required. Longer windows make brief but critical phase coherence difficult to detect. The analysis of nine epileptic rats using EMD detected brief (≥100 ms) but significant synchronies between 7 Hz and 300 Hz. Fourier spectrograms (FIG. 9) of seizures from three rats do show evidence of higher power components in the same coherent frequency range found with the primary analytical technique. But the spread of frequencies of the high amplitude oscillators using Fourier was over a much larger range relative to the target frequencies found with EMD reducing the likelihood of locating brief but significant oscillator synchrony, especially at the lower frequencies, and is likely to be insufficient to determine the critical stimulation parameters as provided by the methodology. Hence, because of the high sensitivity of DBS efficacy on mirroring endogenous rhythms, the use of this more common frequency deconvolution technique appears largely inadequate.

### Location of Stimulation Affects the Efficacy of DBS

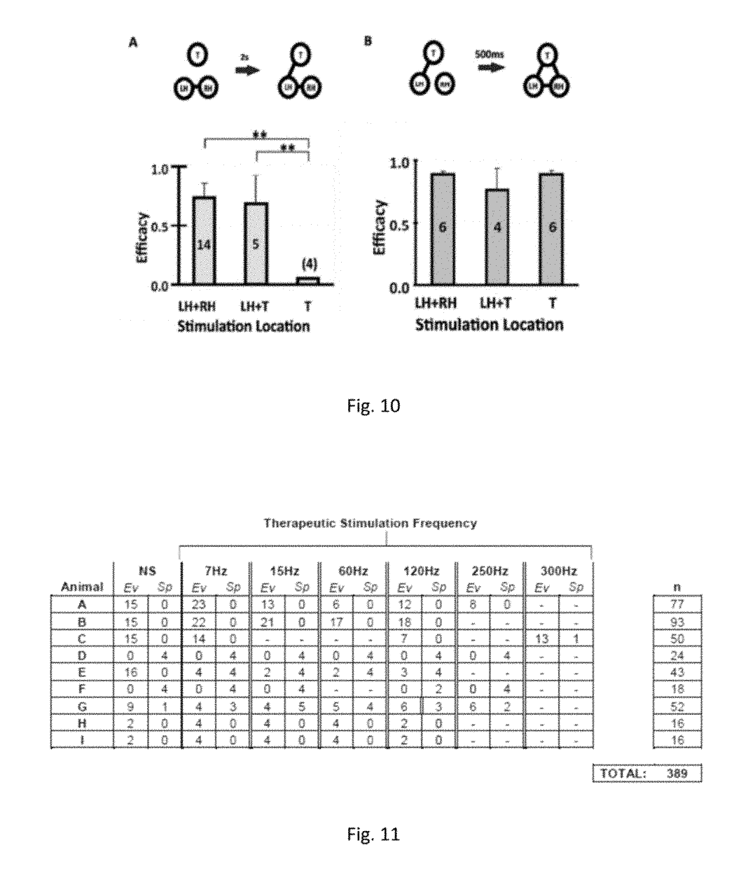

The analysis revealed multi-site dynamics that varied not only in the frequency observed at natural termination of seizures but also the locations of synchronous electrographic activity. FIG. 10 shows different synchrony patterns determined in two rats. When synchrony at natural termination was observed tightly only across the two hippocampi (panel A), stimulation at the target frequency was not effective (i.e., no better than with no therapeutic stimulation) when applied only to the anteromedial thalamus. However, when synchrony in a different subject rapidly occurred across all three structures (panel B), target frequency stimulation applied simultaneously to different variants of these three structures was found to be equally efficacious.

The goal of this research was to investigate whether reproducing endogenous synchrony dynamics observed during natural seizure termination through exogenous electrical stimulation improved the efficacy of reverting epileptic seizures with DBS. A total of 389 seizure events in nine different rat subjects was recorded in this study and tested for the efficacy of stimulation protocols based on endogenous rhythms (see Table, FIG. 11). The key findings of this study were: (1) the efficacy in reverting seizures via DBS was significantly improved when the stimulation frequency matched the target frequency obtained from synchrony analysis, and (2) the efficacy in reverting seizures with DBS greatly depended on matching the locations of multi-site stimulation to those brain regions displaying endogenous synchrony as seizures naturally terminated.

Most studies in the use of DBS for epilepsy treatment derive stimulation parameters by trial and error. When it comes to the stimulation frequency, there exists competing rationale for selecting low, medium, or high. This might explain the varying degrees of success shown in several studies of using DBS to treat recurrent seizures. Since the endogenous dynamics of the seizures were not typically considered in the selection of the DBS frequency, differences in endogenous dynamics between subjects may lead to significantly different efficacy assessments of a single stimulation frequency. The data indicate that the time taken for a seizure to stop was significantly lower when the stimulation frequency matched that from the endogenous synchrony analysis of each subject. The location of the stimulation also appeared to be an important factor when it came to stimulation efficacy. For all subjects, the hippocampus appeared to be an important location for stimulation. Thalamic stimulation alone was less effective than bilateral hippocampal stimulation or a combination of both. One or both hippocampi showed involvement in the synchrony that first emerged as seizures naturally terminated in all subjects. While there was significant difference in the time it took for a seizure to stop between purely hippocampal stimulation and purely thalamic stimulation, a significant difference between bilateral hippocampal stimulation and unilateral (left or right) hippocampal stimulation (p=0.290) was not detected. Because of the stronger commissural connections between both hippocampi in rats than in humans, this sensitivity to sites of stimulation may be even higher when stimulation is applied clinically to human epilepsy patients because of the lower connectivity and hence higher electrical isolation between hemispheres.

The exact nature of the anti-convulsant action of electrical stimulation is not fully understood. Generally accepted theories center around the following hypotheses: (a) preferential release of inhibitory neurotransmitters due to stimulation and/or (b) depolarization block, in which the stimulated neurons are inactivated because of stimulus-induced membrane hyperpolarization. It has also been suggested that stimulation with a high frequency has a lesion-like effect (depolarization block), while low frequency stimulation induces long-term depression (LTD) of stimulated neurons in the peri-electrode space. However, since the target frequencies in some cases were quite low (<20 Hz), it's unlikely that this depolarization block after 10 sec of therapeutic stimulation would have been significant.

### Example III

In this example, an adaptive non-linear analytical methodology was used to extract stimulation frequency and location(s) from endogenous brain dynamics of epilepsy patients, using phase-synchrony and phase-connectivity analysis, as seizures evolve. The method was applied to seizures recorded using depth electrodes implanted in both hippocampus and amygdala in three patients. A reduction in phase-synchrony was observed in all patients around seizure onset. However, phase-synchrony started to gradually increase from mid-ictal and achieved its maximum level at seizure termination. This result suggests that hyper-synchronization of the epileptic network may be a crucial mechanism by which the brain naturally terminates seizure. Stimulation frequency and locations that matched the network phase-synchrony at seizure termination were extracted using phase-connectivity analysis. One patient with temporal lobe epilepsy (TLE) had a stimulation frequency of ˜15 Hz with the stimulation locations confined to the hippocampus. The other two patients with extratemporal lobe epilepsy (ETE) had stimulation frequencies of ˜90 Hz with at least one stimulation location outside of the hippocampus. These results suggest that DBS parameters vary based on the patient's underlying pathology. The methodology provides an algorithm for tuning DBS parameters for individual patients in an effort to increase the clinical efficacy of the therapy.

### Dataset and Methods

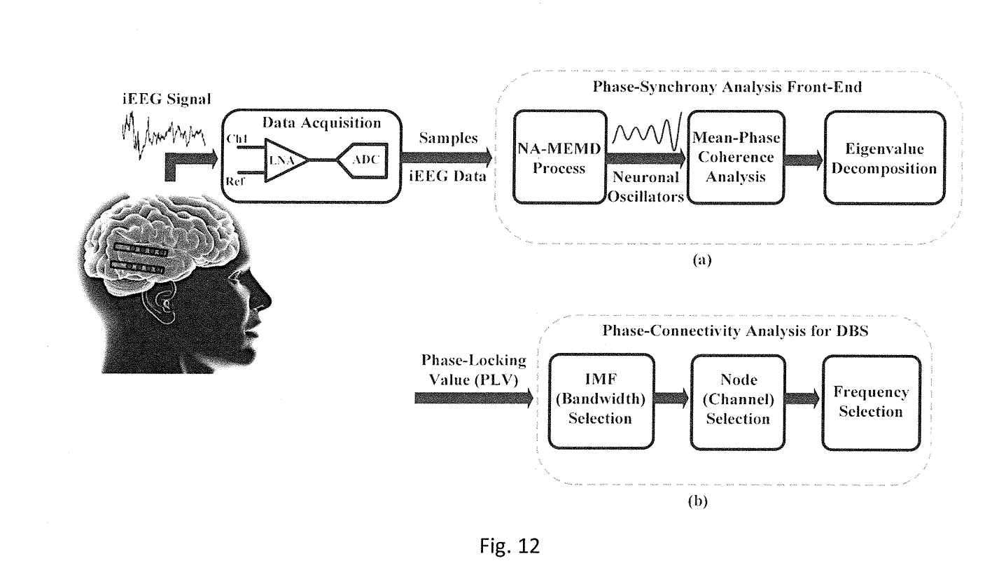

The conceptual framework of the instantaneous phase-synchrony analysis is shown in FIG. 12. Multi-channel iEEG data, recorded from different brain regions of epilepsy patients using implanted depth electrodes, were used as input to the analytical signal processing procedure. A set of finite neuronal oscillators, IMFs, were extracted using NA-MEMD from raw iEEG data. Next, Hilbert transform was performed on the decomposed neuronal oscillators in order to measure their instantaneous phases and frequencies. A combination of mean-phase coherence analysis and eigenvalue decomposition technique was then employed to evaluate the phase-synchrony dynamics among neuronal oscillators as seizures evolved.

FIG. 12 further illustrates the framework of the proposed phase connectivity analysis among neuronal oscillators to extract the frequency and locations of stimulation. This process involved selecting IMF (bandwidth of interest), nodes (locations of stimulation), and finally frequency of stimulation. It should be noted that the aforementioned parameters were extracted to match the maximum level of phase-synchronization observed at seizure termination.

Patients and iEEG Dataset

Multi-channel iEEG data recorded from depth electrodes implanted in three de-identified epilepsy patients were retrieved from the online International Epilepsy Electro-physiology (IEEG) portal. Two patients were diagnosed with extratemporal lobe epilepsy (ETE) with left temporal onset, while the third patient had temporal lobe epilepsy (TLE) with a right temporal seizure focus. All patients were adults undergoing long-term iEEG monitoring in preparation for the pre-surgical evaluation including seizure onset zone (SOZ) localization of their medically refractory epilepsy. Epileptic SOZs and timing of seizures, seizure onset and termination, were clinically determined and annotated by the treating physicians at Mayo Clinic via visual inspection of clear ictal discharges. Further clinical information regarding the analyzed patients as well as their access IDs in the IEEG portal are provided in Table I. The iEEG data were recorded from the patients using depth electrodes with 4 or 8 recording sites, a 500 Hz sampling rate, and a 1-150 Hz bandpass filtering. All epilepsy patients were implanted with two depth electrodes in both amygdala and hippocampal regions of the brain. A digital 60 Hz notch filter was used to eliminate line noise.

TABLE I
PATIENT CLINICAL DATA AND SIGNAL
ACQUISITION INFORMATION

| IEEG Portal | | | |
|---|---|---|---|
| Patient ID | Study 005 | Study 019 | Study 029 |
| Subject | 1 | 2 | 3 |
| Age at Onset | 21 | 31 | 19 |
| Sex | Male | Male | Female |
| No. of Seizures | 6 | 6 | 3 |
| Sampling Rate (Hz) | 500 | 500 | 500 |
| No. of Depth Elec. | 2 × 8 | 2 × 4 | 2 × 4 |
| | contact | contact | contact |
| Seizure Type | SPS | SPS/CPS/GTC | CPS/GTC |
| Epilepsy | TLE | ETE | ETE |

TLE: temporal lobe epilepsy;
ETE: extra temporal lobe epilepsy;
SPS: simple partial seizure;
CPS: complex partial seizure;
GTC: generalized tonic-clonic seizure

### Noise-Assisted Multivariate EMD (NA-MEMD)

EMD is an adaptive, data-driven method of decomposing signals into a set of finite and nearly orthogonal oscillators, called intrinsic mode functions (IMFs). The decomposed IMFs together form the underlying oscillations within a time series. That is, IMFs are truly determined from the dynamics of original signals considering the nature of their underlying components. Therefore, EMD is an appropriate method for the time-frequency analysis of non-linear, non-stationary electro-physiological signals. However, EMD of multi-channel data can result in mode-aliasing and mode-misalignment. An advanced version of the EMD method called NA-MEMD was used to avoid known issues. The original signal is described by the following equation at the end of the decomposition:

$$x ( t ) = {\sum }_{i = 1}^{N} {D}_{i} ( t ) + r ( t ) ,$$

where N is the number of the extracted IMFs and r(t) is the residue. The residue corresponds to a signal whose projections do not contain sufficient extrema to comprise a multi-variate envelope. The lower-index IMFs correspond to fast oscillation modes of the original signal while the extracted higher-index IMFs denote the slower oscillation modes.

NA-MEMD was performed on iEEG data segments to obtain its corresponding IMFs. The instantaneous phases of IMFs were measured using the Hilbert transform.

### Hilbert Analytic Signal Method

Hilbert analytic signal method has been widely utilized to extract instantaneous phase and frequency information from narrowband signals. In order to extract unambiguous phase information, the phase space trace of an analytic signal should possess a single center of rotation. Multi-component or wideband signals yield trajectories in the complex plane with multiple centers of rotation. Therefore, it is essential to individually extract the underlying components of a time series, contributing to the multiple centers of rotation, in order to reach an unambiguous phase reading. The NA-MEMD method provides a proper instantaneous phase determination by separating a wideband signal into its underlying oscillatory components or IMFs. Each one of these IMFs results in a complex plane with a single center of rotation. In this study, NA-MEMD of iEEG signals were performed for each 1-s segment of data prior to the use of the Hilbert transform to measure the instantaneous phase points.

### Mean-Phase Coherence Analysis

In order to determine the strength of phase relationship among the extracted IMFs, the instantaneous phase points of each IMF was represented as a row vector and stacked on top of each other to form an N×M matrix, in which N is the total number of IMFs decomposed from all channels within a 1-s data segment and M denotes the total length of the time series. Next, the bivariate mean-phase coherence matrix, R_{N×N}, was calculated using the following equation:

$${R}_{kp} = \vert \frac{1}{M} {\sum }_{m = 1}^{M} {e}^{[ i ( {φ}_{km} ( t ) - {φ}_{pm} ( t ) ) ]} \vert ,$$

in which i is equal to √−1, M is the total number of samples within the data segment, and Ø(t) is the instantaneous phase of the analytic signal pair indicated by the subscripts k and p. Subscripts k and p iterate from 1 to N and all the values in the R_{N×N} are between zero and one.

### Eigenvalue Decomposition and Synchrony Analysis

Eigenvalue decomposition of the square, bivariate mean-phase coherence matrix was carried out in order to achieve a multi-variate measure for capturing phase-synchrony among all the extracted neuronal oscillators. All the eigenvalues were sorted in ascending order to construct an eigenvalue spectrum. Each eigenvalue indicates how strongly oscillators are phase-correlated in the direction of its associated eigenvector.

The eigenvalue decomposition was performed by solving R_{N×N}vᵢ=λᵢvᵢ, where λᵢ and vᵢ are the obtained eigenvalues and their corresponding eigenvectors, respectively. It is important to note that all the N obtained eigenvalues were real with their sum equal to the total number of the IMFs, as the R_{N×N} matrix was square symmetric. Therefore, any decrease in one of the eigenvalues must be compensated by an increase in the other eigenvalues in order to keep the sum constant. This is the main idea underlying capturing the phase-synchrony between the extracted oscillators. Due to the ascending order between the eigenvalues, a small change in the value of a few higher-index or larger eigenvalues causes a relatively large dynamic change in the value of the majority of lower-index eigenvalues in order to compensate it. Hence, the temporal changes of phase-synchrony are captured with higher sensitivity by focusing on the high percentage of lower-index eigenvalues. The average value of the first 60% lower-index eigenvalues, meanλ_{1:60%}, was computed for each 1-s segment of data to quantify the temporal evolution of phase-synchrony levels.

Computational modeling was described in a previous study to quantify and compare meanλ_{1:60%} value among three epileptic networks with different phase-synchrony level. Simulation results demonstrated that as the phase-synchrony level increases between neuronal oscillators from its minimum to its maximum, the meanλ_{1:60%} value decreases accordingly. Furthermore, the normalized meanλ_{1:60%} values were measured to quantify phase-synchrony levels as seizures evolve in order to magnify the small relative changes of the meanλ_{1:60%} over time. It should be noted that phase-synchrony was observed to increase from mid-ictal towards seizure end and achieved its maximum level at seizure termination in all of the analyzed epilepsy patients.

### IMF (Bandwidth) Selection for DBS

A 10-s window leading up to the seizure termination was selected for phase-connectivity analysis in order to provide adequate timing for evaluating changes in phase relationships. The main purpose of the IMF selection process was to identify which of the extracted IMFs mostly contributed to the hyper-synchronization observed at seizure termination.

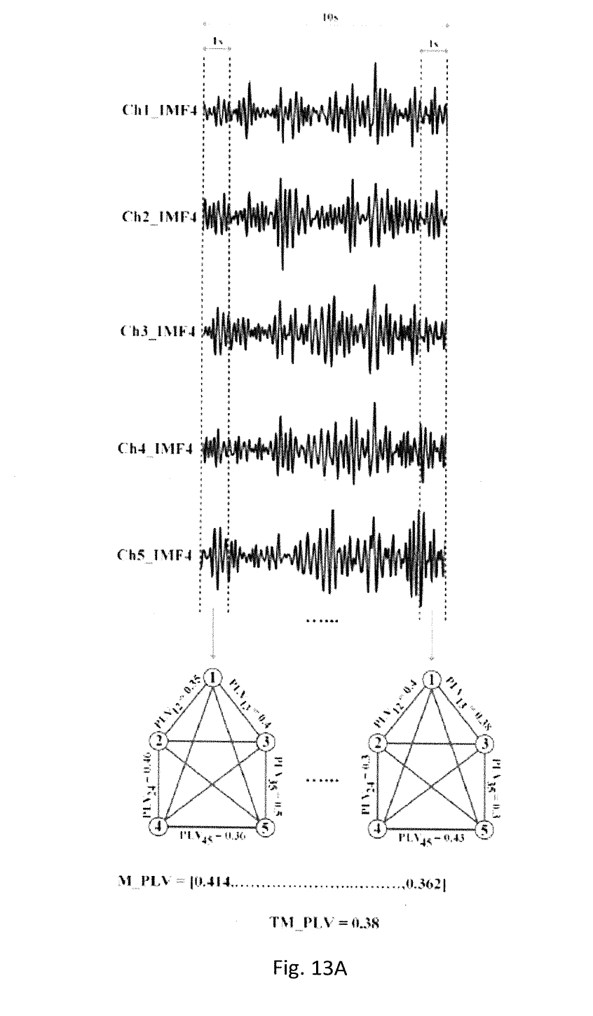

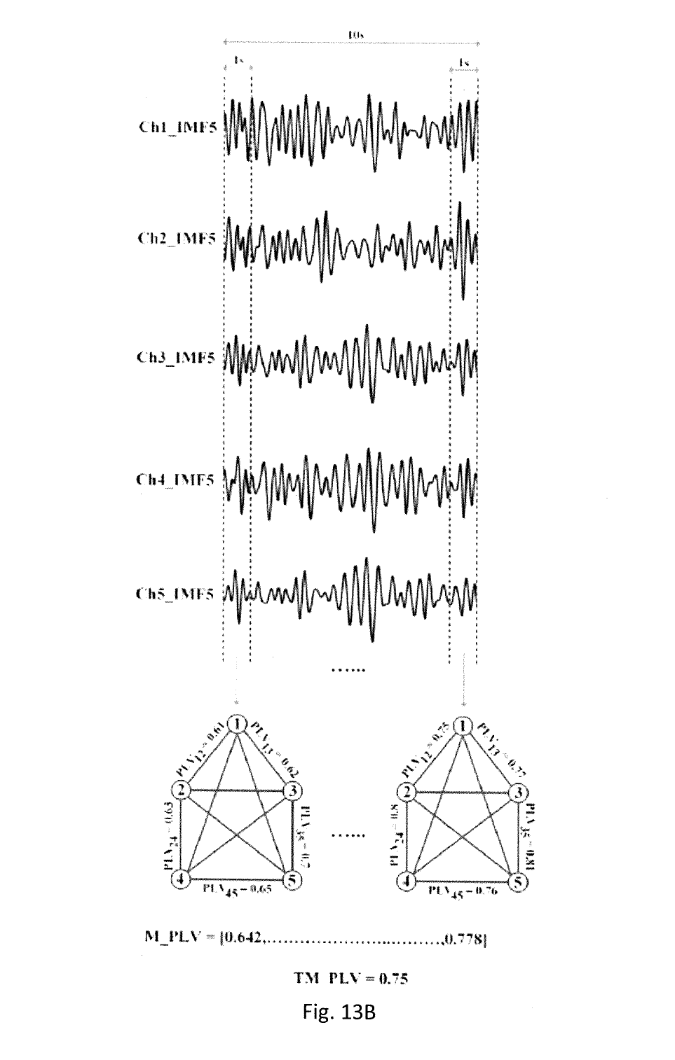

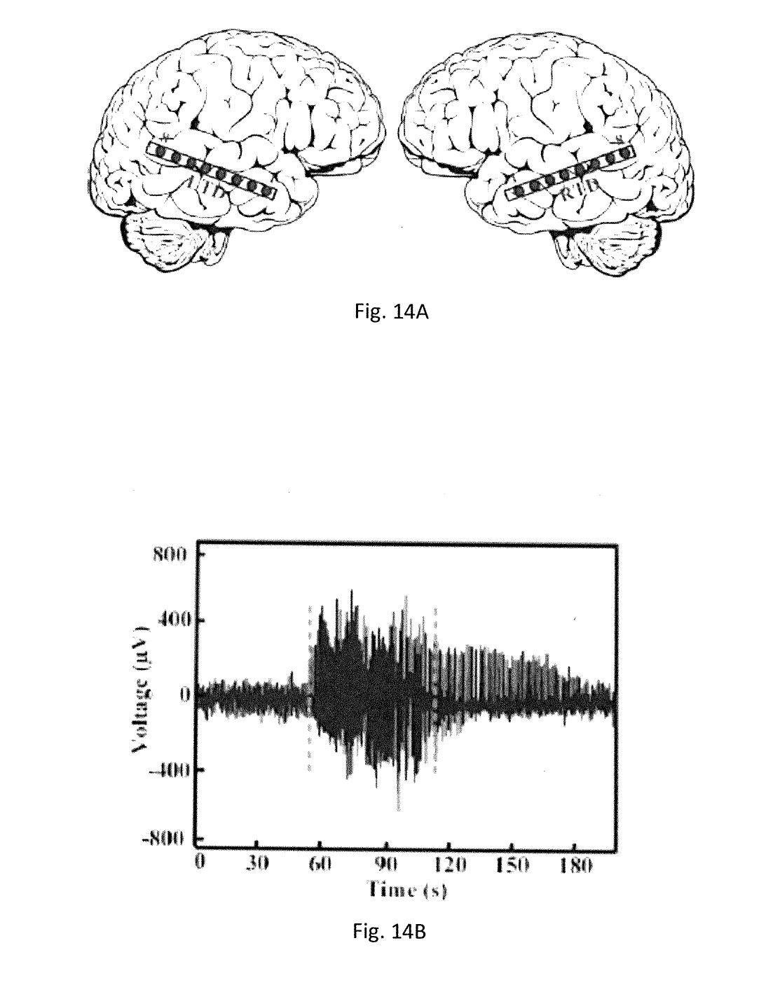

FIGS. 13A and 13B illustrate how phase-connectivity of the extracted IMFs were evaluated. This example displays the comparison between two different IMFs in order to select the one that has higher phase-synchronization within the termination window. The same index IMFs from five iEEG channels were grouped to model the phase-connectivity analysis. FIG. 13A illustrates the fourth neuronal oscillators (IMF4) decomposed from five different iEEG channels within a 10-s window leading up to seizure termination. The bivariate phase-locking value, PLV, was then calculated in each 1-s segment for every unique pairs of channels. The PLV value was measured according to the following equation:

$${PLV}_{vw} = \vert \frac{1}{S} {\sum }_{s = 1}^{S} {e}^{[ i ( {φ}_{vs} ( t ) - {φ}_{ws} ( t ) ) ]} \vert ,$$

in which i is equal to √−1, S is the total number of samples within the data segment, and ϕ(t) is the instantaneous phase of the IMF extracted from channels indicated by subscripts v and w. PLV values can be between zero and one. Circles in FIGS. 13A and 13B represent iEEG channels and PLV values indicate the strength of phase-connection between a pair of channels. For simplicity, only the PLV values of the connections forming the outer edge of the diagram are displayed for the first and last 1-s segments. The average PLV value among all connections, M-PLV, was then calculated for each 1-s segment. Finally, the average M-PLV values were measured for the entire 10-s termination window, which was defined by TM-PLV value. FIG. 13B shows the aforementioned steps on the fifth IMFs (IMF5) extracted from the same five iEEG channels within the 10-s termination window.

As it is shown in FIGS. 13A and 13B, neuronal oscillators within IMF5 resulted in a higher TM-PLV value relative to the ones in IMF4. Therefore, it contributes more into increasing the phase-synchrony level at termination window. The IMF index with the largest TM-PLV value was selected as the one with the highest contribution to the hyper-synchronization observed at seizure termination. It should be noted that the frequency bandwidth of the selected IMF was considered as the tuning range for the frequency of stimulation.

### Node (Channel) Selection for DBS

Once the IMF of interest was identified, the next step was to determine which iEEG channels would be the best candidates for applying DBS. First, the PLV values were calculated in each 1-s segment for every unique pairs of channels, within the selected IMF, for the entire 10-s termination-window. Next, the temporal average of PLV values was measured over the 10-s termination-window for each unique pair. This step results in a L×1 array which L is defined as follows:

$$L = \frac{C ( C - 1 )}{2} ,$$

in which C is the total number of nodes or iEEG channels. Finally, nodes that mostly contributed to the hyper-synchrony observed at seizure termination-window, within the selected IMF, were determined by the following steps:
1. TM-PLV values for the selected IMFs ranged between 0.6 and 0.96 for all seizures among all epilepsy patients. A threshold of the 75^{th} percentile of the PLV values in the L×1 array was found to consistently result in pairs of channels with PLV greater than 0.6 for all seizures. It should be noted that pairs of channels with higher PLV values contribute more to the high phase-synchronization at the network level;
2. A list was formed with all pairs of channels with PLV values exceeding the threshold;
3. The number of times a channel appears in the list (as part of a pair) was defined as its degree of connection. The degree of connection of a channel is indicative of its effectiveness in controlling the network. That is, a node with a high degree of connection would entrain a larger portion of the network resulting in a significant increase in the network phase-synchrony;
4. Channels that appear at least twice (degree of connection greater than one) were considered candidate nodes for DBS. A channel with degree of connections equal to 0 is an isolated node and a channel with degree of connection equal to 1 is an end node. Both of these cases possess minimal potential for driving an epileptic network into a hyper-synchronization; and
5. Channels or nodes that were considered as candidates and consistently appeared among all seizures of a patient were selected as final locations of stimulation.

### Frequency Selection for DBS

The phase of each IMF was measured using the Hilbert transform for each 1-s segment for the entire 10-s termination window. The frequency of each IMF was calculated by taking the time derivative of its instantaneous phases over each 1-s data segment. Finally, the average frequency over the 10-s termination window for each selected node, in the previous section, was measured as the stimulation frequency for that specific channel. The variation in frequency values across the 10-s termination window for any channel was less than 3%.

### Experimental Results

All seizures from the three epilepsy patients were analyzed using the proposed methodology. To deal with the edge effect problem associated with the data segmentation during the NA-MEMD process, 50 samples from neighboring segments were incorporated to both ends of each 1-s segment. However, after executing the NA-MEMD, only the IMFs corresponding to the original data segment were retained. Furthermore, although all IMFs were considered for the phase-synchrony analysis, only IMFs with frequency above 4 Hz were included in the sub-Sequent analysis to determine the stimulation parameters. The IMFs with frequency below 4 Hz did not show any changes in their phase-connectivity with respect to seizures.

### Phase-Connectivity Analysis to Select DBS Parameters in Patient-1

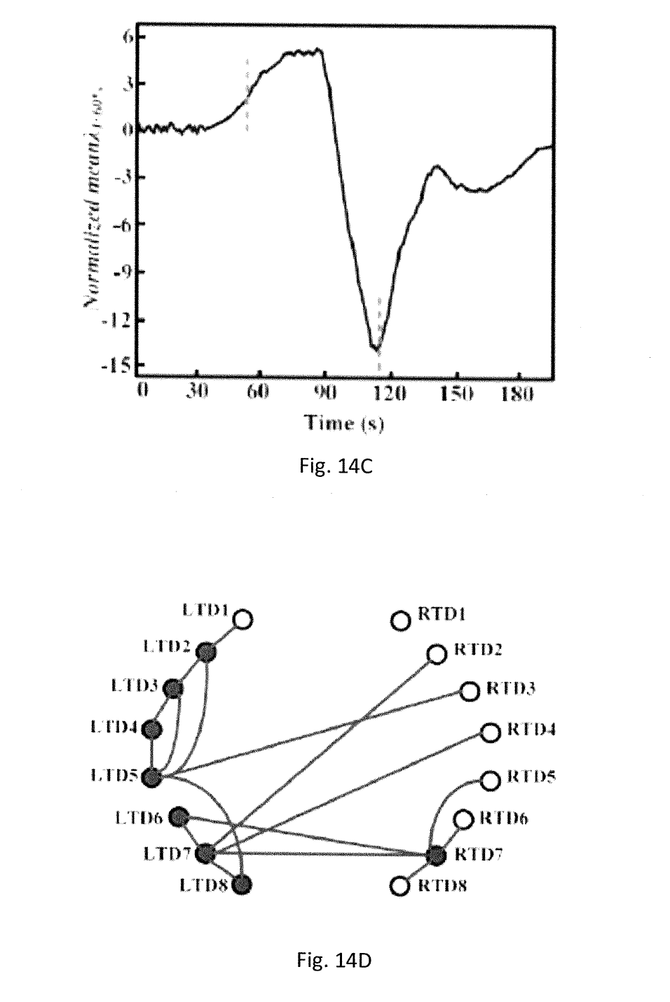

Network phase-synchrony and phase-connectivity analysis were performed on seizures recorded in Patient-1 with TLE in order to determine stimulation frequency and locations. FIG. 14A illustrates the anatomical position of depth electrodes in Patient-1. FIG. 14B shows a 3-min iEEG signal, containing an epileptic seizure, used for this analysis. The dashed lines indicate seizure onset and termination that were clinically determined by treating physicians. Temporal changes in the normalized meanλ_{1:60%} variable, which reflects the evolution of network phase-synchrony, is illustrated in FIG. 14C. The normalized meanλ_{1:60%} value started to increase from about 15-s preceding seizure onset up to almost 30-s after it, which indicates a phase desynchronization among oscillators within this period. However, the synchrony level started to gradually increase toward seizure offset and reached its maximum level at seizure termination. This result suggests that the epileptic network is maximally synchronized at this point. Table II lists the TM-PLV values for all IMFs above the cut-off frequency of 4 Hz for all three patients. The IMF5 was selected as the bandwidth of interest, because it possessed the highest TM-PLV value. It should be noted that IMF5 consistently had the maximum TM-PLV value among all seizures in this patient.

TABLE II
TM-PLV VALUES AT TERMINATION WINDOW
IN DIFFERENT IMFS AMONG ALL PATIENTS

| | TM-PLV | | | | | |
|---|---|---|---|---|---|---|
| Subject | IMF 1 | IMF 2 | IMF 3 | IMF 4 | IMF 5 | IMF 6 |
| 1 | 0.31 | 0.28 | 0.26 | 0.38 | 0.75 | 0.35 |
| 2 | 0.48 | 0.69 | 0.41 | 0.43 | 0.48 | 0.45 |
| 3 | 0.73 | 0.93 | 0.80 | 0.62 | 0.34 | 0.53 |

FIG. 14D displays the connections between unique pairs of channels for the selected IMF, (IMF5). The circles denote the recording sites on the depth electrodes implanted in Patient-1. The lines drawn between pairs of channels indicate the ones with PLV value above the 75th percentile threshold. The filled circles are the candidate nodes for DBS. This analysis was performed on all recorded seizures for Patient-1. The final nodes, candidate nodes appeared in all seizures for this patient were LTD5, LTD7 and RTD7. It should be noted that the final nodes for DBS correlated well with the channels possessing the highest degree of connections. These channels appeared to be hubs and thereby are well suited for synchronizing the epileptic network. All of these channels were located in the hippocampus and the measured frequency of them within the termination window was approximately 15 Hz. The measured frequency was the same among all seizures for this patient and was chosen as the proposed stimulation frequency.

### Phase-Connectivity Analysis to Select DBS Parameters in Patient-2

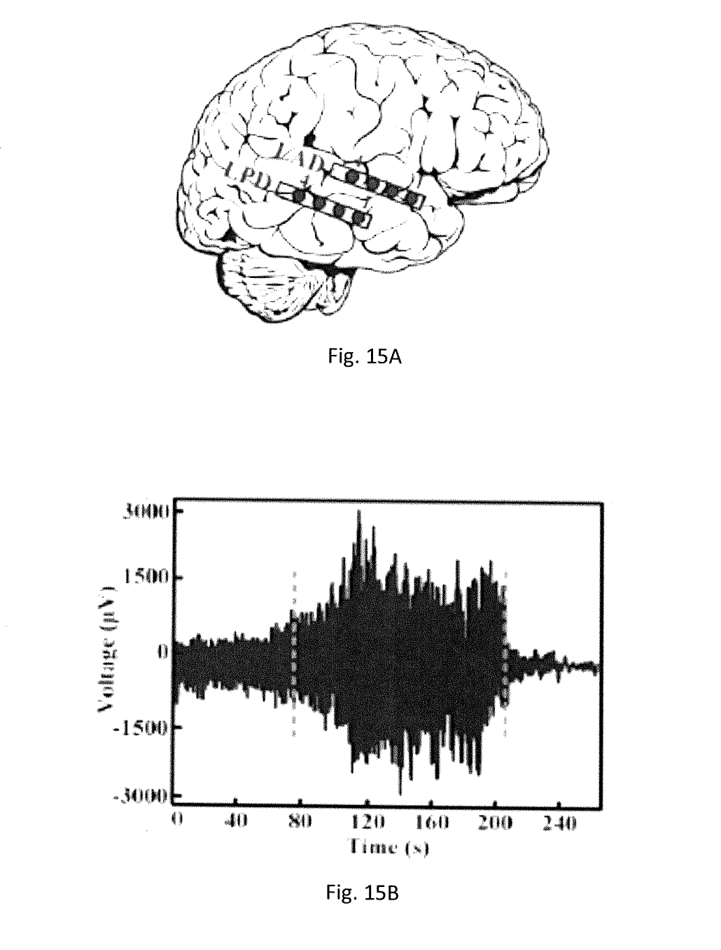

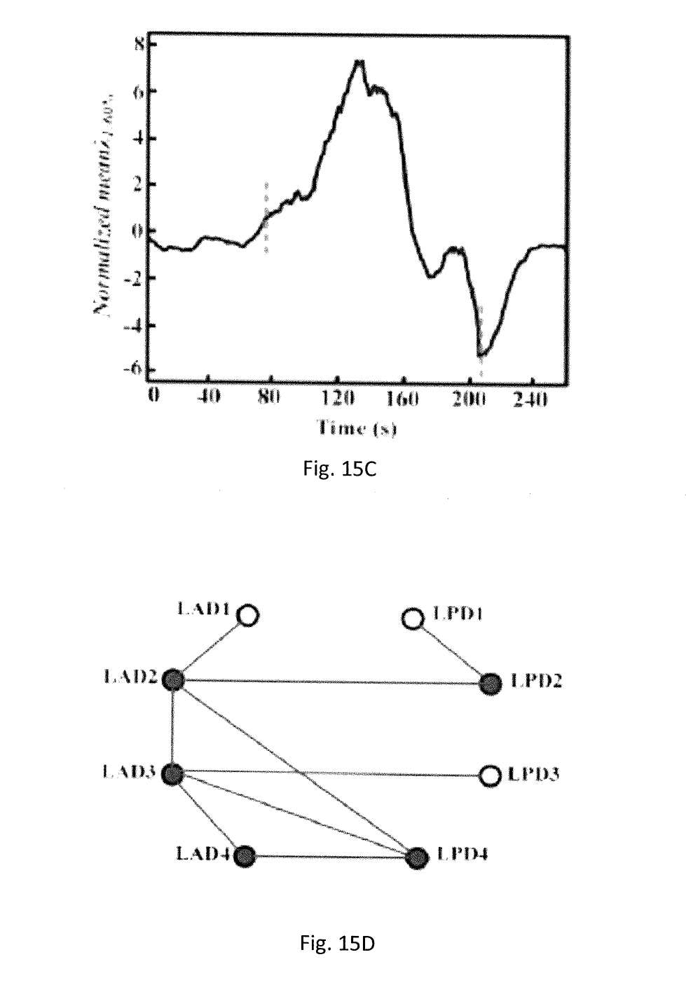

The same analysis described in the previous section was carried out for Patient-2. FIG. 15 displays the phase-synchrony and phase-connectivity dynamics for an iEEG data containing an epileptic seizure in Patient-2 with ETE. FIG. 15A shows the anatomical position of the depth electrodes in the patient. A 4-min iEEG signal utilized for the phase-synchrony and phase-connectivity analysis, is shown in FIG. 15B. Dashed lines indicate seizure onset and offset. FIG. 15C exhibits the normalized meanλ_{1:60%} obtained from the phase-synchrony analysis. The phase-synchrony pattern was similar to the one observed in Patient-1. The network achieved the maximum phase-synchrony level at seizure termination. The bandwidth of interest was measured to be the second IMF (IMF2) as it had the highest TM-PLV value, which is reported in Table II. The selected IMF was the same among all seizures analyzed in this patient. FIG. 15D displays the candidate nodes for DBS for a recorded seizure in Patient-2 using the same procedure applied to Patient-1. The final nodes for DBS were identified as LAD2 and LAD3. The final nodes for DBS overlapped well with the obtained candidate nodes with the highest degree of connections in the network. These sites appeared to be hubs in the network and further supported selecting these channels as simulation sites for DBS. Both of these channels were located in the amygdala and the measured frequency of them within the termination window was about 90 Hz. The calculated frequency was the same among all seizures for this patient and was selected as the proposed stimulation frequency.

### Phase-Connectivity Analysis for the Selection of DBS Parameters in Patient-3

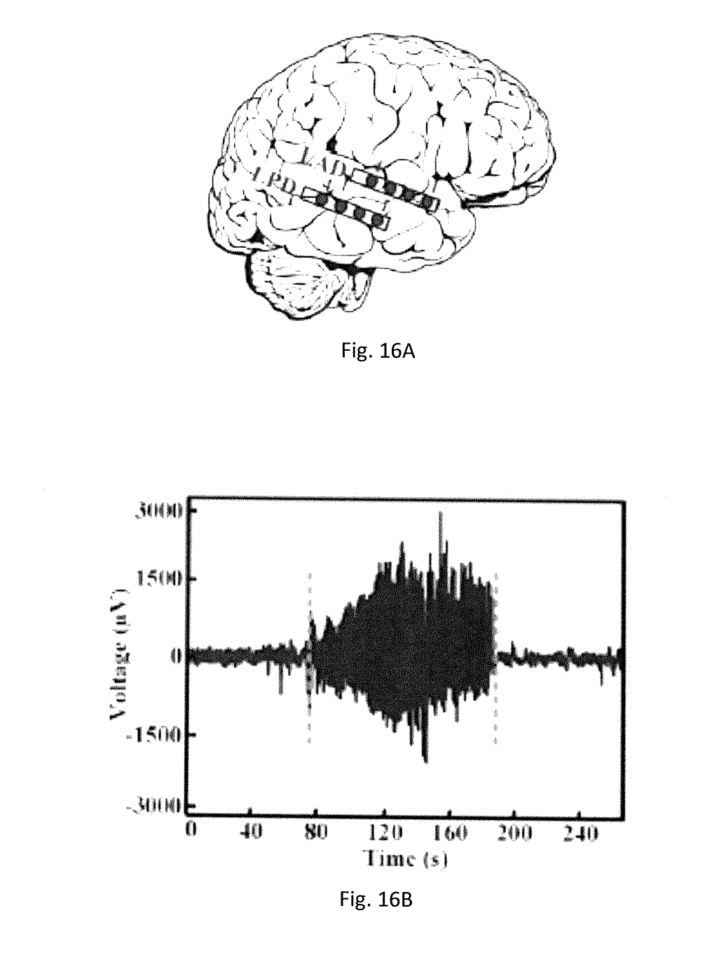

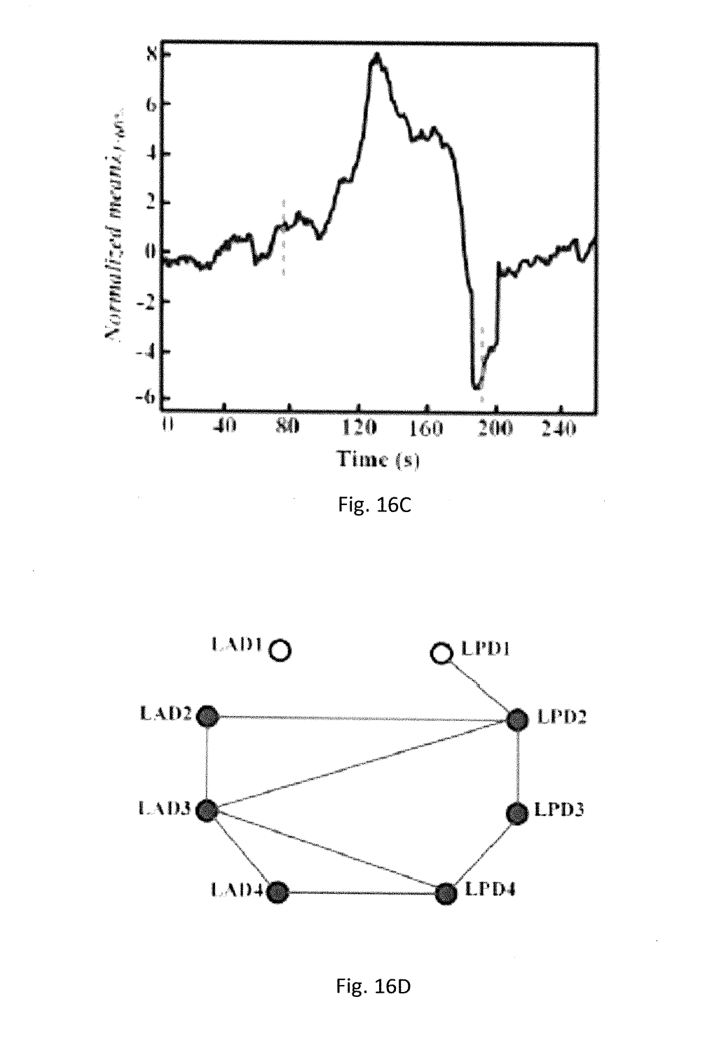

FIG. 16 exhibits the phase-synchrony and phase-connectivity dynamics for an iEEG signal containing a seizure in Patient-3 with ETE. FIG. 16A illustrates the anatomical position of the depth electrodes in this patient. FIG. 16B shows a 4-min iEEG signal utilized for this analysis. Dashed lines denote seizure onset and offset. The normalized meanλ_{1:60%} variable obtained from the phase-synchrony analysis is shown in FIG. 16C. A phase desynchronization was observed from ictal onset to mid-ictal. However, the synchrony level started to gradually increase from mid-ictal towards ictal offset and achieved its maximum level at seizure termination. As shown in Table II, the second IMF (IMF2) was obtained as the bandwidth of interest as it had the highest TM-PLV value among the other extracted IMFs. Moreover, IMF2 was consistently obtained as the IMF with the maximum TM-PLV value among all seizures analyzed in this patient. FIG. 16D displays the candidate nodes for DBS for one of the recorded seizures in Patient-3 using the analysis reported in the method section. For Patient-3, the final nodes for stimulation were identified as LAD3 and LPD2. The final nodes for DBS overlapped well with the candidate nodes with high degree of connections. Besides, these channels appeared to be hubs in the network with respect to their degree of connections. One of the channels was located in the amygdala and the other was located in the hippocampus. The frequency of these nodes within the termination window was obtained to be approximately 90 Hz. This frequency was the same among all seizures recorded in this patient, and thereby was selected as the proposed stimulation frequency.

### Discussion

In this example, the adaptive non-linear analytical methodology merges both phase-synchrony and phase-connectivity analysis in order to extract stimulation frequency and locations that match the endogenous brain dynamics of patients as seizures naturally terminate. This method provides an algorithm for tuning DBS parameters in order to develop a DBS protocol that can be delivered upon detection of sub-sequent seizures in an effort to terminate them immediately. Applying DBS using the proposed closed-loop approach could potentially improve the clinical efficacy of the therapy. The analysis proposed in this example are divided into two major components; namely, (1) Phase-synchrony analysis in order to determine how network synchrony changes as seizures evolve, and (2) Phase-connectivity analysis in order to select IMF (bandwidth of interest), channels (locations of stimulation), and finally frequency of electrical stimulation. These parameters were extracted to match the maximum level of phase-synchrony observed at seizure termination in order to reproduce hyper-synchrony.

The first step in the phase-synchrony analysis was the decomposition of wideband electrophysiological signals into a set of finite, narrow-band neuronal oscillators using NA-MEMD. The instantaneous phases of the oscillators or IMFs were then calculated using the Hilbert transform. Next, the eigenvalue decomposition of the square, bivariate mean-phase coherence matrix, R_{N×N}, was performed to access the phase-synchrony dynamics between IMFs. The normalized mean value of the first 60% lower-index eigenvalues, meanλ_{1:60%}, was measured and reported for each second to determine changes in the network's synchrony level as seizures evolve. In all patients, a decrease in phase-synchrony was observed from seizure onset to mid-ictal. The phase-desynchronization period was followed by a gradual increase in phase-synchronization level as seizure progressed and reached its maximum level at seizure termination. The hyper-synchrony at seizure termination provided the basis for the phase-connectivity analysis. The phase-connectivity analysis used bivariate phase-locking value, PLV, to locate the neuronal oscillators that contribute the most to the hyper-synchronization observed at seizure offset. This was achieved by finding the neuronal oscillators that were highly synchronized in the 10-s window leading up to seizure termination.

Both the obtained frequency and locations of stimulation representing the hyper-synchronization at termination window varied among the patients analyzed in this study. It should be noted that the extracted stimulation frequency and locations were stable among all seizures in all patients. Patient-1 with TLE had identified locations in the hippocampus while the other two patients with ETE had at least one of their site outside of the hippocampus. The frequency of stimulation obtained in Patient-1 was lower than the other two patients. Patient-1 had bilateral hippocampal sclerosis while the other two patients possessed no lesions. These results suggest that the underlying pathology may contribute to the variance in frequency and locations of stimulation obtained in epilepsy patients. Most DBS therapy using available FDA-approved devices utilize pre-determined stimulation parameters such as stimulation frequency and locations for a cohort of patients. In clinical studies, patients showing more than 50% reduction in seizure frequency in comparison to baseline are labelled as responders and those that do not, are categorized as non-responders. The therapeutic DBS protocol described in this invention can increase the responder's rate using current DBS devices by accounting for patient's specific brain dynamics when determining stimulation parameters. A recent study that investigated hippocampal stimulation in patients with drug-resistant mesial temporal lobe epilepsy (MTLE) found individual variation in the optimal DBS frequency. Low frequency hippocampal stimulation was effective (more than 50% reduction in seizure frequency) in MTLE patients with hippocampal sclerosis, while it was ineffective (˜20% reduction in seizure frequency) in patients with no lesions in the hippocampus. Switching stimulation frequency in a patient with no lesion to a high frequency resulted in greater reduction (up to 55%) in seizure frequency relative to the baseline level. Furthermore, several studies have reported the efficacy of high frequency DBS in reducing seizure frequency for epilepsy patients. On the other hand, other studies have suggested that low frequency stimulation is more efficacious in reducing seizure frequency. Computational results of this exemplary analysis also supported the difference in stimulation frequency and locations that matched the hyper-synchrony at seizures termination among epilepsy patients.

In addition to providing stimulation parameters based on patient's brain dynamics, another important feature of closed-loop stimulation is the ability to deliver electrical stimulation in response to seizure detection. In all seizures recorded from patients, there is a decrease in the network's phase-synchrony level a few seconds before seizure onset. This decrease in phase-synchrony may be utilized as a potential trigger in implantable neuromodulation devices for applying electrical stimulation in order to terminate epileptic seizures.

A main aim of this example was to extract DBS stimulation frequency and locations that drive the epileptic network into hyper-synchrony at natural seizure termination. This DBS protocol can be delivered upon seizure detection to artificially revert the phase desynchronization dynamics observed around seizure onset in order to terminate it immediately.

Thus, the invention provides methods of treating seizures by applying individualized, closed-loop stimulation protocols for preventing or terminating epileptic seizures. The analysis of this invention is adaptive and can take into consideration the endogenous brain dynamics of patients, which leads to personalizing and optimizing DBS therapy for them. The methods can be implemented by software implemented protocols on current commercial neurostimulators to determine appropriate synchrony locations and/or termination frequencies, and then automatically apply one or more appropriate frequencies upon detecting a further seizure event.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. A method of preventing or terminating seizures, comprising:
determining neuronal synchrony between at least two sites of a brain of a patient;
stimulating the brain with at least two implanted electrodes, each implanted in a different one of at least two regions of the brain, with a predetermined seizure termination frequency determined from frequencies measured across a seizure occurrence in the at least two regions of the brain.

2. The method of claim 1, wherein the stimulation comprises deep brain stimulation before or during a seizure at the at least two sites.

3. The method of claim 1, wherein the neuronal synchrony between the at least two sites of the brain is determined from brain sites having a highest coherence there between, and the stimulating occurs at the at least two sites of the brain.

4. The method of claim 1, further comprising:
identifying neuronal synchronies between pairs of the plurality of sites; and
electrically applying a frequency selected from the neuronal synchronies to the brain.

5. The method of claim 4, wherein the frequency is determined from a neuronal synchrony indicating higher coherence.

6. The method of claim 4, further comprising detecting a potential or actual seizure occurrence, wherein the frequency is electrically applied to the brain upon the detection of the potential or actual seizure occurrence.

7. The method of claim 1, wherein the frequency promotes synchronization between structures in the at least two regions of the brain to preempt or terminate a seizure.

8. The method of claim 1, wherein the electrodes are actuated by an implanted neurostimulator including a power source.

9. A non-transitory computer readable storage medium storing code executable on an implantable neurostimulator to perform the method according to claim 1.

10. A method of preventing or terminating seizures, comprising:
monitoring for and/or determining neuronal synchrony between a plurality of sites of a brain of a patient;
comparing measurements from and between at least pairs of the plurality of sites to determine at least a pair of sites with a higher electrical coherence than one or more others of the at least pairs;
determining a frequency at the higher electrical coherence; and
stimulating the brain at the frequency.

11. The method of claim 10, wherein the stimulation occurs at the pair of sites.

12. The method of claim 10, wherein the higher the coherence between the at least pairs, the higher the synchronic connectivity between the at least pairs.

13. The method of claim 10, further comprising identifying neuronal synchronies between the at least pairs of the plurality of sites; wherein the frequency is selected from the neuronal synchronies.

14. The method of claim 13, wherein the frequency is determined from a neuronal synchrony indicating the highest electrical coherence.

15. The method of claim 13, further comprising detecting a potential or actual seizure occurrence, wherein the frequency is electrically applied to the brain upon the detection of the potential or actual seizure occurrence.

16. An apparatus for preventing or terminating seizures, comprising:
a neurostimulator including a stimulation generator and a power supply connected to the stimulation generator, wherein the stimulation generator generates electrical stimulation for and through at least two electrodes implantable within a brain; and
a control protocol on a non-transitory recordable medium in executable combination with the stimulation generator and adapted to stimulate at least two regions of the brain with a frequency to emulate neuronal synchrony, wherein the frequency comprises a predetermined seizure termination frequency predetermined from seizure frequencies measured across a seizure occurrence in the at least two regions of the brain.

17. The apparatus of claim 16, wherein the frequency is determined from measurements from and between pairs of the plurality of sites within the brain, and determined from a pair of sites with higher electrical coherence than one or more others of the pairs.

18. The apparatus of claim 17, wherein the predetermined seizure termination frequency is determined by the neurostimulator and electrodes.

19. The apparatus of claim 17, further comprising a detector adapted to determine neuronal synchrony between pairs of the plurality of sites within the brain.

20. The apparatus of claim 19, wherein the detector determines a potential or actual seizure occurrence, and the frequency is electrically applied to the brain upon the detection of the potential or actual seizure occurrence.
